(19) United States
(12) Patent Application Publication
(10) Pub. No.: US 2026/0232796 A1
(43) Pub. Date: Aug. 13, 2026

(54) COMBINATION THERAPIES WITH AN ANTI-FACTOR XI/FACTOR XIA ANTIBODY

(71) Applicant: Anthos Therapeutics, Inc., Boston, MA (US)

(72) Inventors: Debra A. FREEDHOLM, Cambridge, MA (US); Daniel M. BLOOMFIELD, Belmar, NJ (US); Royston J. GLASSPOOL, Cambridge, MA (US); Jonathan E. FREEMAN, Cambridge, MA (US); Yasser KHDER, Rosenau (FR)

(21) Appl. No.: 18/987,711

(22) Filed: Dec. 19, 2024

Related U.S. Application Data
(63) Continuation of application No. PCT/US2023/068956, filed on Jun. 23, 2023.
(60) Provisional application No. 63/355,319, filed on Jun. 24, 2022.

(51) Int. Cl. A61K 39/395; A61K 31/519; A61K 31/616; A61K 38/48; A61K 39/00; A61P 7/02

(52) U.S. Cl. CPC A61K 39/3955; A61K 31/519; A61K 31/616; A61K 38/4846; A61P 7/02; C12Y 304/21021; A61K 2039/505; A61K 2039/545

(57) ABSTRACT

This disclosure relates to dosage regimens for combination therapies comprising anti-Factor XI and/or activated Factor XI (Factor XIa) antibodies, or antigen-binding fragments thereof, e.g., for use in the treatment of thromboembolic disorders or related conditions.

### CROSS-REFERENCE TO RELATED APPLICATIONS

This application is a continuation of International (PCT) Patent Application No. PCT/US2023/068956, filed on Jun. 23, 2023, which claims the benefit of and priority to U.S. Provisional Patent Application No. 63/355,319, filed on Jun. 24, 2022, the disclosure of which is hereby incorporated by reference in its entirety for all purposes.

### SEQUENCE LISTING

This application contains a Sequence Listing XML, which has been submitted electronically and is hereby incorporated by reference in its entirety. Said XML copy, created on Dec. 18, 2024, is named ATD-012WOC1_SL.XML and is 52,230 bytes in size.

### FIELD OF THE DISCLOSURE

The present disclosure relates to dosage regimens for combination therapies comprising anti-Factor XI and/or activated Factor XI (Factor XIa) antibodies, or antigen-binding fragments thereof, e.g., for use in the treatment of thromboembolic disorders or related conditions.

### BACKGROUND

There exists a high unmet medical need for safer therapies to reduce thromboembolic complications such as stroke, systemic embolism, cognitive decline and mortality, with comparable or improved efficacy to that exhibited by existing therapies, and with a lower risk of bleeding.

Factor XI (FXI) is a serine protease functioning both in the intrinsic and extrinsic coagulation pathways. Factor XI exists in the zymogen form as a homodimer; upon cleavage of the peptide bond at R369-1370, Factor XI is activated (Factor XIa, FXIa). FXI plays a minor role in normal hemostasis in a high tissue factor environment but does play a key role in thrombosis. Genetic Factor XI deficiency is associated with decreased incidence of ischemic stroke and venous thromboembolic events (Salomon et al. (2008); Salomon, et al. (2011) Thromb Haemost.; 105:269-73). Bleeding manifestations in subjects with Factor XI deficiency are infrequent, often mild, result from injury or trauma, and very rarely affect critical organs (Salomon et al. (2011)).

Antibodies that bind Factor XI and/or Factor XIa have been studied. For example, WO 2016/207858 describes one such anti-Factor XI and/or Factor XIa antibody, disclosed herein in Table 1 as Antibody 1. The present disclosure adds to these developments and provides further clinical methods, including combination dosage regimens, to treat patients with specific thromboembolic disorders with desired safety and efficacy.

### SUMMARY

The present disclosure provides combination therapies with anti-Factor XI and/or Factor XIa antibodies or antigen-binding fragments thereof, or pharmaceutical formulations comprising the same.

Accordingly, in one aspect, provided herein is a method of reversing an anticoagulant effect of an isolated anti-Factor XI (FXI) and/or anti-activated Factor XI (FXIa) antibody, or an antigen-binding fragment thereof, in a subject being administered the isolated antibody or antigen-binding fragment thereof at a dose of about 150 mg, wherein the method comprises administering to the subject a therapeutically effective amount of recombinant activated factor VII (rFVIIa), thereby reversing the anticoagulant effect.

In some embodiments, the therapeutically effective amount of rFVIIa is a low dose. In certain embodiments, the therapeutically effective amount of rFVIIa is between 0.5 and 1 μg/mL. In some embodiments, the therapeutically effective amount of rFVIIa is administered once. In some embodiments, the therapeutically effective amount of rFVIIa is administered more than once. In some embodiments, the therapeutically effective amount of rFVIIa is administered as needed.

In some embodiments, the administering the therapeutically effective amount of rFVIIa prolongs clotting time (CT) relative to CT prior to the administering the therapeutically effective amount. In some embodiments, the administering the therapeutically effective amount of rFVIIa prolongs clot formation time (CFT) relative to CFT prior to the administering the therapeutically effective amount. In certain embodiments, the CT or CFT is determined by rotational thromboelastometry (ROTEM) in a whole blood assay. In certain embodiments, the whole blood assay is ex vivo or in vitro.

In certain embodiments, the method further comprises applying one of the following to the subject: (i) fluid replacement using colloids, crystalloids, human plasma or plasma proteins such as albumin; (ii) transfusion with packed red blood or whole blood; (iii) administration of fresh frozen plasma (FFP), prothrombin complex concentrates (PCC), activated PCC (APCC), such as, factor VIII inhibitor, or (iv) an anti-idiotypic antibody against the isolated anti-FXI and/or anti-FXIa antibody or antigen-binding fragment thereof.

In another aspect, provided herein is a method of treating a disease or disorder in a subject in need thereof, the method comprising administering to the subject a dose of about 150 mg of an isolated anti-Factor XI (FXI) and/or anti-activated Factor XI (FXIa) antibody, or an antigen-binding fragment thereof, and administering (a) about 1 mg/mL aspirin or (b) about 3 μM ticagrelor.

In some embodiments, the aspirin or ticagrelor is administered prior to the administering of the antibody or antigen-binding fragment thereof.

In some embodiments, the aspirin or ticagrelor is administered concurrently with the administering of the antibody or antigen-binding fragment thereof.

In some embodiments, the aspirin or ticagrelor is administered after the administering of the antibody or antigen-binding fragment thereof.

In some embodiments, the administering of the antibody or antigen-binding fragment thereof does not affect platelet aggregation in the subject as compared to the subject treated with only aspirin or ticagrelor. In certain embodiments, the platelet aggregation is measured by impedance platelet aggregometry. In certain embodiments, the platelet aggregation is induced by collagen, adenosine 5′-diphosphate (ADP), or thrombin receptor activating peptide-6 (TRAP-6).

In certain embodiments, the platelet aggregation is determined ex vivo or in vitro.

In some embodiments, the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

In some embodiments of the above aspects, the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37 (or the amino acid sequence of KNY); and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37 (or the amino acid sequence of KNY); and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

In some embodiments, the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

In some embodiments, the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

In some embodiments, the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto. In certain embodiments, the antibody comprises a heavy chain comprising an amino acid sequence of SEQ ID NO: 31 and a light chain comprising an amino acid sequence of SEQ ID NO: 41.

In some embodiments, the antibody is a human monoclonal antibody.

In some embodiments, the antibody is a human IgG1 isotype.

In some embodiments, the antibody comprises D265A and P329A substitutions in the Fc domain.

In some embodiments, the antibody or antigen-binding fragment thereof is administered intravenously.

In some embodiments, the antibody or antigen-binding fragment thereof is administered subcutaneously.

In some embodiments, the antibody or antigen-binding fragment thereof is administered in a drug delivery formulation comprising a histidine buffer at a concentration of about 20 mM. In some embodiments, the antibody or antigen-binding fragment thereof is administered in a drug delivery formulation comprising sucrose at a concentration of about 220 mM. In some embodiments, the antibody or antigen-binding fragment thereof is administered in a drug delivery formulation comprising polysorbate 20 at a concentration of about 0.04%. In some embodiments, the antibody or antigen-binding fragment thereof is administered in a drug delivery formulation at pH 5.5.

In some embodiments, when the antibody or antigen-binding fragment thereof is administered in an intravenous drug delivery formulation, the intravenous drug delivery formulation further comprises about 5% glucose.

In some embodiments, the subject is afflicted with or at risk of developing a thromboembolic disorder. In some embodiments, the thromboembolic disorder is selected from the group consisting of atrial fibrillation or atrial flutter, transient ischemic attack, ischemic stroke, thromboembolic stroke, hemorrhagic stroke, venous thromboembolism (VTE), pediatric VTE, systemic embolism, non-central nervous systemic embolism, myocardial infarction, deep vein thrombosis, Severe Protein S deficiency, cerebrovascular accident, and cancer.

In some embodiments, a first dose of the antibody or antigen-binding fragment thereof is administered intravenously and a second dose of the antibody or antigen-binding fragment is administered subcutaneously. In certain embodiments, the method further comprises one or more additional doses of the antibody or antigen-binding fragment thereof administered subcutaneously following the administering of the second dose.

In some embodiments the antibody or antigen-binding fragment thereof is administered about once a month.

Other embodiments and details of the disclosure are presented herein below.

### BRIEF DESCRIPTION OF THE DRAWINGS

FIGS. 1A-1B depict in vitro platelet aggregation in donor whole blood supplemented with vehicle, Antibody 1, or abciximab following induction with collagen (FIG. 1A) or TRAP-6 (FIG. 1B).

FIGS. 2A-2B depict in vitro effect of Antibody 1 combined with aspirin (ASA) on whole blood platelet aggregometry induced with 3.2 μg/mL collagen (FIG. 2A) or with 0.5 mM arachidonic acid (FIG. 2B) Error bars indicate one standard deviation.

FIGS. 3A-3B depict in vitro effect of Antibody 1 combined with ticagrelor on whole blood platelet aggregometry induced with 6.4 μM ADP (FIG. 3A) or with 32 μM TRAP6 (FIG. 3B). Error bars indicate one standard deviation.

FIGS. 4A-4B depict comparative in vitro effect of Antibody 1 and rFVIIa on the rotational thromboelastometry (ROTEM) parameters clotting time (CT, provided in seconds) (FIG. 4A) and clot formation time (CFT, provided in seconds) (FIG. 4B) of human blood. Values are shown as mean and standard deviation percent values express the difference compared to vehicle alone (baseline). Dotted lines delimit the normal reference interval. * indicates p<0.05 with respect to vehicle alone (baseline).

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “FXI protein,” “FXI antigen,” and “FXI” are used interchangeably, and refers to the Factor XI protein in different species. Factor XI is the mammalian plasma coagulation Factor XI, a glycoprotein present in human plasma at a concentration of 25-30 nM as a zymogen that when converted by limited proteolysis to an active serine protease, participates in the intrinsic pathway of blood coagulation.

The terms “FXIa protein,” “FXIa antigen,” and “FXIa”, are used interchangeably, and refers to the activated FXI protein in different species. The zymogen Factor XI is converted into its active form, the coagulation Factor XIa (FXIa), either via the contact phase of blood coagulation or through thrombin-mediated activation on the platelet surface. During this activation of factor XI, an internal peptide bond is cleaved in each of the two chains, resulting in the activated factor XIa, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor II/Thrombin activation. For example, human FXI has the sequence as set out in Table 1 (SEQ ID NO:1) and has been described in previous reports and literature (Mandle R J Jr, et al. (1979) Blood; 54 (4): 850; NCBI Reference Sequence: AAA51985).

In the context of this present disclosure, the terms “FXI” and “FXIa” (and the like) include mutants and variants of the natural FXI and FXIa protein, respectively, which have substantially the same amino acid sequence as that of the native primary structure (amino acid sequence) described in the above-mentioned reports.

The term “catalytic domain,” “serine protease catalytic domain,” and similar terms as used herein, means amino acids Ile370 to Val607, as counted from the Glu1 at the N-terminus of the mature protein that is in circulation. It can also be described as residues 388-625 at the C-terminus of FXI. As used herein, the term “active site” means the catalytic triad comprised of the amino acids His413, Asp462 and Ser557. (Bane and Gailani (2014) Drug Disc. 19 (9)).

The term “antibody” as used herein means a whole antibody and any antigen binding fragment (e.g., “antigen-binding portion”) or single chain thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. In some specific aspects, an antibody can be a monoclonal antibody, human antibody, humanized antibody, camelid antibody, or chimeric antibody.

Antibodies can be of any isotype (e.g., IgG, IgE, IgM, IgD, IgA and IgY), class (e.g., IgG1, IgG2, IgG3, IgG4, IgAQ1 and IgA2) or subclass.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “drug delivery formulation” or “intravenous drug delivery formulation” refers to a pharmaceutical formulation comprising the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans. In certain embodiments, the subject is a human. As used herein “primate subject” is inclusive of both human and non-human primates. In certain embodiments, the subject is a baboon model of thrombosis, described, for example, in Gruber et al. Blood, 1989 Feb. 15; 73 (3): 639-42 and Crosby et al. Arterioscler Thromb Vasc Biol, 2013 July; 33 (7): 1670-8.

A “thromboembolic disorder,” or similar terms as used herein, refer to any number of conditions or diseases in which the intrinsic and/or common coagulation pathways are aberrantly activated or are not naturally deactivated (e.g., without therapeutic means). These conditions include but are not limited to thromboembolic stroke and other types of stroke of ischemic origin, atrial fibrillation, stroke prevention in atrial fibrillation (SPAF), deep vein thrombosis, venous thromboembolism, and pulmonary embolism. These can also include prevention and treatment of catheter-related thrombosis (e.g., Hickman catheter in oncology patients) in which catheters become thrombosed, and extracorporeal membrane oxygenation (ECMO), in which the tubing and oxygenation membrane develops clots.

A “thromboembolic disorder” or similar terms as used herein, can also refer to any number of the following, which the anti-FXI and/or FXIa antibodies or antigen binding fragments thereof of the present disclosure can be used to prevent or treat:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
venous thromboembolism, wherein the subject is a pediatric subject (pediatric VTE);
cerebral and cardiovascular events in secondary prevention after transient ischemic attack (TIA) or non-disabling stroke and prevention of thromboembolic events in heart failure with sinus rhythm;
hemorrhagic stroke;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins or arteries like catheter, pacemaker wires, synthetic arterial grafts; mechanical or biological heart valves or left ventricular assist device;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
non-central nervous systemic embolism (non-CNS systemic embolism);
thrombosis in patients with invasive and non-invasive cancer malignancies (e.g., CAT);
thrombosis over an indwelling catheter;
thrombosis and thromboembolism in severely ill patients;
cardiac thrombosis and thromboembolism, including but not limited to cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
thromboembolism in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease; and
thrombosis and thromboembolism in patients undergoing hemodialysis and in patients undergoing extra-corporal membrane oxygenation.

As used herein, the term “trough” or “trough level” refers to the lowest concentration reached by a drug before the next dose of the drug is administered. In certain embodiments, inhibition of Factor XI/Factor XIa at trough is greater than about 50% (e.g., greater than about 60%, greater than about 70%, greater than about 80%, or greater than about 90%). In certain embodiments, inhibition of Factor XI/Factor XIa at trough is greater than about 80%. In certain embodiments, inhibition of Factor XI/Factor XIa at trough is greater than about 90%.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By “therapeutic benefit,” what is meant is eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

In certain embodiments of the methods described herein, the subject is treatment naïve, i.e., has never received any form of anticoagulant therapy prior to treatment with an anti-Factor XI/XIa antibody described herein, e.g., Antibody 1. In certain embodiments of the methods described herein, the subject has received a stable treatment of a recommended dose of a new oral anticoagulant (NOAC), e.g., prior to treatment with an anti-Factor XI/XIa antibody described herein, e.g., Antibody 1. In certain embodiments, the subject has received a direct oral anticoagulant (DOAC) e.g., prior to treatment with an anti-Factor XI/XIa antibody described herein, e.g., Antibody 1. In certain embodiments, the subject has received a Vitamin K antagonist (VKA) e.g., prior to treatment with an anti-Factor XI/XIa antibody described herein, e.g., Antibody 1.

As used herein, the term “vial” refers to a container that holds the drug product. In some embodiments, the vial may be a vial, a bag, a pen, or a syringe. In some embodiments, the vial may be a vial, e.g., a glass vial.

As used herein, the term “drug product” refers to an anti-Factor XI/XIa antibody described herein, e.g., Antibody 1 as disclosed in Table 1, and excipients, e.g., a histidine buffer, a sugar, and a polysorbate.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Anti-Factor XI and/or Activated Factor XI (Factor XIa) Antibodies

In some embodiments, the present disclosure provides pharmaceutical formulations comprising antibodies that bind FXI and/or FXIa protein (e.g., human, rabbit, cynomolgus monkey, and baboon FXI and/or FXIa), wherein the antibodies comprise a heavy chain variable domain (VH) having an amino acid sequence of SEQ ID NOs: 9 or 29, the formulations comprise a histidine buffer; a sugar or sugar alcohol; and a polysorbate, and the pH of the formulation is between pH 5.0 to 6.0. In certain embodiments, the antibodies comprise a VH having an amino acid sequence of SEQ ID NO:29.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides a pharmaceutical formulations of antibodies that specifically bind to a FXI and/or FXIa protein, wherein the antibodies comprise a VH CDR having an amino acid sequence of any one of the VH CDRs listed in Table 1, infra, the formulations comprise a histidine buffer; a sugar or sugar alcohol; and a polysorbate; and the pH of the formulation is between pH 5.0 to 6.0. In particular, the present disclosure provides pharmaceutical formulations of antibodies that specifically bind to a FXI and/or FXIa protein (e.g., human, rabbit, cynomolgus monkey, and baboon FXI and/or FXIa), wherein the antibodies comprise (or alternatively, consist of) one, two, three, or more VH CDRs having an amino acid sequence of any of the VH CDRs listed in Table 1, infra, the formulations comprise a histidine buffer; a sugar or sugar alcohol; and a polysorbate; and the pH of the formulation is between pH 5.0 to 6.0. (see PCT International Patent Application No. PCT/IB2016/053790 filed on Jun. 24, 2016, and published as WO2016/207858, which is hereby incorporated by reference in its entirety).

In some embodiments, the present disclosure provides pharmaceutical formulations of antibodies that specifically bind to a FXI/FXIa protein, said antibodies comprising a light chain variable domain (VL) having an amino acid sequence of SEQ ID NOs: 19 or 39, for use in the methods described herein (e.g., methods for treating a subject afflicted with or at risk of developing a thromboembolic disorder), the formulations comprise a histidine buffer; a sugar or sugar alcohol; and a polysorbate; and the pH of the formulation is between pH 5.0 to 6.0. In certain embodiments, the antibodies comprise a VL having an amino acid sequence of SEQ ID NO: 39.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides pharmaceutical formulations of antibodies that specifically bind to a FXI and/or FXIa protein (e.g., human, rabbit, cynomolgus monkey, and baboon FXI and/or FXIa), for use in the methods described herein (e.g., methods for treating a subject afflicted with or at risk of developing a thromboembolic disorder), the antibodies comprising a VL CDR having an amino acid sequence of any one of the VL CDRs listed in Table 1, infra; the formulations comprise a histidine buffer; a sugar or sugar alcohol; and a polysorbate; and the pH of the formulation is between pH 5.0 to 6.0. The antibodies that specifically bind to an FXIa protein (e.g., human, rabbit, cynomolgus monkey, and baboon FXI and/or FXIa), may comprise (or alternatively, consist of) one, two, three or more VL CDRs having an amino acid sequence of any of the VL CDRs listed in Table 1, infra.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In some embodiments, other antibodies for use in the methods described herein (e.g., methods for treating a subject afflicted with or at risk of developing a thromboembolic disorder) include amino acids that have been mutated, yet have at least 60, 70, 80, 85, 90 or 95 percent identity in the CDR regions with the CDR regions depicted in the sequences described in Table 1. In some embodiments, the antibodies include mutant amino acid sequences wherein no more than 1, 2, 3, 4 or 5 amino acids have been mutated in the CDR regions when compared with the CDR regions depicted in the sequence described in Table 1.

TABLE 1
Examples of FXI/FXIa Antibodies, Fabs and FXI/FXIa Proteins

| | Sequence | |
|---|---|---|
| | Identifier | |
| | (SEQ ID | |
| Sequence Description | NO:) | Amino acid or polynucleotide sequence |
| Human FXIa full- | 1 | MIFLYQVVHFILFTSVSGECVTQLLKDTCFEGGDIT |
| length protein | | TVFTPSAKYCQVVCTYHPRCLLFTFTAESPSEDPTR |
| sequence (NCBI | | WFTCVLKDSVTETLPRVNRTAAISGYSFKQCSHQI |
| Reference Sequence: | | SACNKDIYVDLDMKGINYNSSVAKSAQECQERCT |
| AAA51985) | | DDVHCHFFTY ATRQFPSLEHRNICLLKHTQTGTPT |
| | | RITKLDKVVSGFSLKSCALSNLACIRDIFPNTVFAD |
| | | SNIDSVMAPDAFVSGRICTHHPGCLFFTFFSQEWP |
| | | KESQRNLCLLKTSESGLPSTRIKKSKALSGFSLQSC |
| | | RHSIPVFCHSSFYHDTDFLGEELDIVAAKSHEACQ |
| | | KLCTNAVRCQFFTYTPAQASCNEGKGKCYLKLSS |
| | | NGSPTKILHGRGGISGYTLRLCKMDNECTTKIKPRI |
| | | VGGTASVRGEWPWQVTLHTTSPTQRHLCGGSIIG |
| | | NQWILTAAHCFYGVESPKILRVYSGILNQSEIKEDT |
| | | SFFGVQEIIIHDQYKMAESGYDIALLKLETTVNYTD |
| | | SQRPICLPSKGDRNVIYTDCWVTGWGYRKLRDKI |
| | | QNTLQKAKIPLVTNEECQKRYRGHKITHKMICAG |
| | | YREGGKDACKGDSGGPLSCKHNEVWHLVGITSW |
| | | GEGCAQRERPGVYTNVVEYVDWILEKTQAV |
| Human FXIa full- | 2 | AGGCACACAGGCAAAATCAAGTTCTACATCTGT |
| length nucleotide | | CCCTGTGTATGTCACTTGTTTGAATACGAAATAA |
| sequence (NCBI | | AATTAAAAAAATAAATTCAGTGTATTGAGAAAG |
| Reference Sequence: | | CAAGCAATTCTCTCAAGGTATATTTCTGACATAC |
| NM_000128.3) | | TAAGATTTTAACGACTTTCACAAATATGCTGTAC |
| | | TGAGAGAGAATGTTACATAACATTGAGAACTAG |
| | | TACAAGTAAATATTAAAGTGAAGTGACCATTTC |
| | | CTACACAAGCTCATTCAGAGGAGGATGAAGACC |
| | | ATTTTGGAGGAAGAAAAGCACCCTTATTAAGAA |
| | | TTGCAGCAAGTAAGCCAACAAGGTCTTTTCAGG |
| | | ATGATTTTCTTATATCAAGTGGTACATTTCATTT |
| | | TATTTACTTCAGTTTCTGGTGAATGTGTGACTCA |
| | | GTTGTTGAAGGACACCTGCTTTGAAGGAGGGGA |
| | | CATTACTACGGTCTTCACACCAAGCGCCAAGTA |
| | | CTGCCAGGTAGTCTGCACTTACCACCCAAGATG |
| | | TTTACTCTTCACTTTCACGGCGGAATCACCATCT |
| | | GAGGATCCCACCCGATGGTTTACTTGTGTCCTGA |
| | | AAGACAGTGTTACAGAAACACTGCCAAGAGTGA |
| | | ATAGGACAGCAGCGATTTCTGGGTATTCTTTCAA |
| | | GCAATGCTCACACCAAATAAGCGCTTGCAACAA |
| | | AGACATTTATGTGGACCTAGACATGAAGGGCAT |
| | | AAACTATAACAGCTCAGTTGCCAAGAGTGCTCA |
| | | AGAATGCCAAGAAAGATGCACGGATGACGTCCA |
| | | CTGCCACTTTTTCACGTACGCCACAAGGCAGTTT |
| | | CCCAGCCTGGAGCATCGTAACATTTGTCTACTGA |
| | | AGCACACCCAAACAGGGACACCAACCAGAATA |
| | | ACGAAGCTCGATAAAGTGGTGTCTGGATTTTCA |
| | | CTGAAATCCTGTGCACTTTCTAATCTGGCTTGTA |
| | | TTAGGGACATTTTCCCTAATACGGTGTTTGCAGA |
| | | CAGCAACATCGACAGTGTCATGGCTCCCGATGC |
| | | TTTTGTCTGTGGCCGAATCTGCACTCATCATCCC |
| | | GGTTGCTTGTTTTTTACCTTCTTTTCCCAGGAATG |
| | | GCCCAAAGAATCTCAAAGAAATCTTTGTCTCCTT |
| | | AAAACATCTGAGAGTGGATTGCCCAGTACACGC |
| | | ATTAAAAAGAGCAAAGCTCTTTCTGGTTTCAGTC |
| | | TACAAAGCTGCAGGCACAGCATCCCAGTGTTCT |
| | | GCCATTCTTCATTTTACCATGACACTGATTTCTT |
| | | GGGAGAAGAACTGGATATTGTTGCTGCAAAAAG |
| | | TCACGAGGCCTGCCAGAAACTGTGCACCAATGC |
| | | CGTCCGCTGCCAGTTTTTTACCTATACCCCAGCC |
| | | CAAGCATCCTGCAACGAAGGGAAGGGCAAGTGT |
| | | TACTTAAAGCTTTCTTCAAACGGATCTCCAACTA |
| | | AAATACTTCACGGGAGAGGAGGCATCTCTGGAT |
| | | ACACATTAAGGTTGTGTAAAATGGATAATGAGT |
| | | GTACCACCAAAATCAAGCCCAGGATCGTTGGAG |
| | | GAACTGCGTCTGTTCGTGGTGAGTGGCCGTGGC |
| | | AGGTGACCCTGCACACAACCTCACCCACTCAGA |
| | | GACACCTGTGTGGAGGCTCCATCATTGGAAACC |
| | | AGTGGATATTAACAGCCGCTCACTGTTTCTATGG |
| | | GGTAGAGTCACCTAAGATTTTGCGTGTCTACAGT |
| | | GGCATTTTAAATCAATCTGAAATAAAAGAGGAC |
| | | ACATCTTTCTTTGGGGTTCAAGAAATAATAATCC |
| | | ATGATCAGTATAAAATGGCAGAAAGCGGGTATG |
| | | ATATTGCCTTGTTGAAACTGGAAACCACAGTGA |
| | | ATTACACAGATTCTCAACGACCCATATGCCTGCC |
| | | TTCCAAAGGAGATAGAAATGTAATATACACTGA |
| | | TTGCTGGGTGACTGGATGGGGGTACAGAAAACT |
| | | AAGAGACAAAATACAAAATACTCTCCAGAAAGC |
| | | CAAGATACCCTTAGTGACCAACGAAGAGTGCCA |
| | | GAAGAGATACAGAGGACATAAAATAACCCATA |
| | | AGATGATCTGTGCCGGCTACAGGGAAGGAGGGA |
| | | AGGACGCTTGCAAGGGAGATTCGGGAGGCCCTC |
| | | TGTCCTGCAAACACAATGAGGTCTGGCATCTGG |
| | | TAGGCATCACGAGCTGGGGCGAAGGCTGTGCTC |
| | | AAAGGGAGCGGCCAGGTGTTTACACCAACGTGG |
| | | TCGAGTACGTGGACTGGATTCTGGAGAAAACTC |
| | | AAGCAGTGTGAATGGGTTCCCAGGGGCCATTGG |
| | | AGTCCCTGAAGGACCCAGGATTTGCTGGGAGAG |
| | | GGTGTTGAGTTCACTGTGCCAGCATGCTTCCTCC |
| | | ACAGTAACACGCTGAAGGGGCTTGGTGTTTGTA |
| | | AGAAAATGCTAGAAGAAAACAAACTGTCACAA |
| | | GTTGTTATGTCCAAAACTCCCGTTCTATGATCGT |
| | | TGTAGTTTGTTTGAGCATTCAGTCTCTTTGTTTTT |
| | | GATCACGCTTCTATGGAGTCCAAGAATTACCAT |
| | | AAGGCAATATTTCTGAAGATTACTATATAGGCA |
| | | GATATAGCAGAAAATAACCAAGTAGTGGCAGTG |
| | | GGGATCAGGCAGAAGAACTGGTAAAAGAAGCC |
| | | ACCATAAATAGATTTGTTCGATGAAAGATGAAA |
| | | ACTGGAAGAAAGGAGAACAAAGACAGTCTTCA |
| | | CCATTTTGCAGGAATCTACACTCTGCCTATGTGA |
| | | ACACATTTCTTTTGTAAAGAAAGAAATTGATTGC |
| | | ATTTAATGGCAGATTTTCAGAATAGTCAGGAAT |
| | | TCTTGTCATTTCCATTTTAAAATATATATTAAAA |
| | | AAAATCAGTTCGAGTAGACACGAGCTAAGAGTG |
| | | AATGTGAAGATAACAGAATTTCTGTGTGGAAGA |
| | | GGATTACAAGCAGCAATTTACCTGGAAGTGATA |
| | | CCTTAGGGGCAATCTTGAAGATACACTTTCCTGA |
| | | AAAATGATTTGTGATGGATTGTATATTTATTTAA |
| | | AATATCTTGGGAGGGGAGGCTGATGGAGATAGG |
| | | GAGCATGCTCAAACCTCCCTAAGACAAGCTGCT |
| | | GCTGTGACTATGGGCTCCCAAAGAGCTAGATCG |
| | | TATATTTATTTGACAAAAATCACCATAGACTGCA |
| | | TCCATACTACAGAGAAAAAACAATTAGGGCGCA |
| | | AATGGATAGTTACAGTAAAGTCTTCAGCAAGCA |
| | | GCTGCCTGTATTCTAAGCACTGGGATTTTCTGTT |
| | | TCGTGCAAATATTTATCTCATTATTGTTGTGATC |
| | | TAGTTCAATAACCTAGAATTTGAATTGTCACCAC |
| | | ATAGCTTTCAATCTGTGCCAACAACTATACAATT |
| | | CATCAAGTGTG |
| Antibody 2 | | |
| HCDR1 (Kabat) | 3 | TAAMS |
| HCDR2 (Kabat) | 4 | GISGSGSSTYYADSVKG |
| HCDR3 (Kabat) | 5 | ELSYLYSGYYFDY |
| HCDR1 (Chothia) | 6 | GFTFSTA |
| HCDR2 (Chothia) | 7 | SGSGSS |
| HCDR3 (Chothia) | 8 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 9 | QVQLLESGGGLVQPGGSLRLSCAASGFTESTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 10 | CAGGTGCAATTGCTGGAAAGCGGCGGTGGCCTG |
| | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| Heavy Chain | 11 | QVQLLESGGGLVQPGGSLRLSCAASGFTESTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPEAAGG |
| | | PSVFLFPPKPKDTLMISRTPEVTCVVVDVSHEDPEV |
| | | KFNWYVDGVEVHNAKTKPREEQYNSTYRVVSVL |
| | | TVLHQDWLNGKEYKCKVSNKALPAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 12 | CAGGTGCAATTGCTGGAAAGCGGCGGTGGCCTG |
| Chain | | GTGCAGCCGGGTGGCAGCCTGCGTCTGAGCTGC |
| | | GCGGCGTCCGGATTCACCTTTTCTACTGCTGCTA |
| | | TGTCTTGGGTGCGCCAGGCCCCGGGCAAAGGTC |
| | | TCGAGTGGGTTTCCGGTATCTCTGGTTCTGGTTC |
| | | TTCTACCTACTATGCGGATAGCGTGAAAGGCCG |
| | | CTTTACCATCAGCCGCGATAATTCGAAAAACAC |
| | | CCTGTATCTGCAAATGAACAGCCTGCGTGCGGA |
| | | AGATACGGCCGTGTATTATTGCGCGCGTGAACT |
| | | GTCTTACCTGTACTCTGGTTACTACTTCGATTAC |
| | | TGGGGCCAAGGCACCCTGGTGACTGTTAGCTCA |
| | | GCCTCCACCAAGGGTCCATCGGTCTTCCCCCTGG |
| | | CACCCTCCTCCAAGAGCACCTCTGGGGGCACAG |
| | | CGGCCCTGGGCTGCCTGGTCAAGGACTACTTCC |
| | | CCGAACCGGTGACGGTGTCGTGGAACTCAGGCG |
| | | CCCTGACCAGCGGCGTGCACACCTTCCCGGCTG |
| | | TCCTACAGTCCTCAGGACTCTACTCCCTCAGCAG |
| | | CGTGGTGACCGTGCCCTCCAGCAGCTTGGGCAC |
| | | CCAGACCTACATCTGCAACGTGAATCACAAGCC |
| | | CAGCAACACCAAGGTGGACAAGAGAGTTGAGC |
| | | CCAAATCTTGTGACAAAACTCACACATGCCCAC |
| | | CGTGCCCAGCACCTGAAGCAGCGGGGGGACCGT |
| | | CAGTCTTCCTCTTCCCCCCAAAACCCAAGGACAC |
| | | CCTCATGATCTCCCGGACCCCTGAGGTCACATGC |
| | | GTGGTGGTGGACGTGAGCCACGAAGACCCTGAG |
| | | GTCAAGTTCAACTGGTACGTGGACGGCGTGGAG |
| | | GTGCATAATGCCAAGACAAAGCCGCGGGAGGA |
| | | GCAGTACAACAGCACGTACCGGGTGGTCAGCGT |
| | | CCTCACCGTCCTGCACCAGGACTGGCTGAATGG |
| | | CAAGGAGTACAAGTGCAAGGTCTCCAACAAAGC |
| | | CCTCCCAGCCCCCATCGAGAAAACCATCTCCAA |
| | | AGCCAAAGGGCAGCCCCGAGAACCACAGGTGT |
| | | ACACCCTGCCCCCATCCCGGGAGGAGATGACCA |
| | | AGAACCAGGTCAGCCTGACCTGCCTGGTCAAAG |
| | | GCTTCTATCCCAGCGACATCGCCGTGGAGTGGG |
| | | AGAGCAATGGGCAGCCGGAGAACAACTACAAG |
| | | ACCACGCCTCCCGTGCTGGACTCCGACGGCTCCT |
| | | TCTTCCTCTACAGCAAGCTCACCGTGGACAAGA |
| | | GCAGGTGGCAGCAGGGGAACGTCTTCTCATGCT |
| | | CCGTGATGCATGAGGCTCTGCACAACCACTACA |
| | | CGCAGAAGAGCCTCTCCCTGTCTCCGGGTAAA |
| LCDR1 (Kabat) | 13 | SGSSSNIGSNDVS |
| LCDR2 (Kabat) | 14 | KNYNRPS |
| LCDR3 (Kabat) | 15 | SAWDQRQFDVV |
| LCDR1 (Chothia) | 16 | SSSNIGSND |
| LCDR2 (Chothia) | 17 | KNY |
| LCDR3 (Chothia) | 18 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 19 | DIVLTQPPSVSGAPGQRVTISCSGSSSNIGSNDVSW |
| | | YQQLPGTAPKLLIYKNYNRPSGVPDRESGSKSGTS |
| | | ASLAITGLQAEDEADYYCSAWDQRQFDVVFGGGT |
| | | KLTVL |
| DNA encoding VL | 20 | GATATCGTGCTGACCCAGCCGCCGAGCGTGAGC |
| | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTA |
| Light Chain | 21 | DIVLTQPPSVSGAPGQRVTISCSGSSSNIGSNDVSW |
| | | YQQLPGTAPKLLIYKNYNRPSGVPDRESGSKSGTS |
| | | ASLAITGLQAEDEADYYCSAWDQRQFDVVFGGGT |
| | | KLTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 22 | GATATCGTGCTGACCCAGCCGCCGAGCGTGAGC |
| Chain | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTTG |
| | | TGTTTGGCGGCGGCACGAAGTTAACCGTCCTAG |
| | | GTCAGCCCAAGGCTGCCCCCTCGGTCACTCTGTT |
| | | CCCGCCCTCCTCTGAGGAGCTTCAAGCCAACAA |
| | | GGCCACACTGGTGTGTCTCATAAGTGACTTCTAC |
| | | CCGGGAGCCGTGACAGTGGCCTGGAAGGCAGAT |
| | | AGCAGCCCCGTCAAGGCGGGAGTGGAGACCACC |
| | | ACACCCTCCAAACAAAGCAACAACAAGTACGCG |
| | | GCCAGCAGCTATCTGAGCCTGACGCCTGAGCAG |
| | | TGGAAGTCCCACAGAAGCTACAGCTGCCAGGTC |
| | | ACGCATGAAGGGAGCACCGTGGAGAAGACAGT |
| | | GGCCCCTACAGAATGTTCA |
| Antibody 1 | | |
| HCDR1 (Kabat) | 23 | TAAMS |
| HCDR2 (Kabat) | 24 | GISGSGSSTYYADSVKG |
| HCDR3 (Kabat) | 25 | ELSYLYSGYYFDY |
| HCDR1 (Chothia) | 26 | GFTFSTA |
| HCDR2 (Chothia) | 27 | SGSGSS |
| HCDR3 (Chothia) | 28 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTESTAAMS |
| HCDR2 (Combined) | 4 | GISGSGSSTYYADSVKG |
| HCDR3 (Combined) | 5 | ELSYLYSGYYFDY |
| VH | 29 | QVQLLESGGGLVQPGGSLRLSCAASGFTESTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSS |
| DNA encoding VH | 30 | CAGGTGCAGCTGCTGGAATCAGGCGGCGGACTG |
| | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | C |
| Heavy Chain | 31 | QVQLLESGGGLVQPGGSLRLSCAASGFTESTAAMS |
| | | WVRQAPGKGLEWVSGISGSGSSTYYADSVKGRFT |
| | | ISRDNSKNTLYLQMNSLRAEDTAVYYCARELSYL |
| | | YSGYYFDYWGQGTLVTVSSASTKGPSVFPLAPSSK |
| | | STSGGTAALGCLVKDYFPEPVTVSWNSGALTSGV |
| | | HTFPAVLQSSGLYSLSSVVTVPSSSLGTQTYICNVN |
| | | HKPSNTKVDKRVEPKSCDKTHTCPPCPAPELLGGP |
| | | SVFLFPPKPKDTLMISRTPEVTCVVVAVSHEDPEV |
| | | KFNWYVDGVEVHNAKTKPREEQYNSTYRVVSVL |
| | | TVLHQDWLNGKEYKCKVSNKALAAPIEKTISKAK |
| | | GQPREPQVYTLPPSREEMTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLS |
| | | PGK |
| DNA encoding Heavy | 32 | CAGGTGCAGCTGCTGGAATCAGGCGGCGGACTG |
| Chain | | GTGCAGCCTGGCGGTAGCCTGAGACTGAGCTGC |
| | | GCTGCTAGTGGCTTCACCTTTAGCACCGCCGCTA |
| | | TGAGCTGGGTTCGACAGGCCCCAGGGAAAGGCC |
| | | TCGAGTGGGTCTCAGGGATTAGCGGTAGCGGCT |
| | | CTAGCACCTACTACGCCGATAGCGTGAAGGGCC |
| | | GGTTCACTATCTCTAGGGATAACTCTAAGAACA |
| | | CCCTGTACCTGCAGATGAATAGCCTGAGAGCCG |
| | | AGGACACCGCCGTCTACTACTGCGCTAGAGAGC |
| | | TGAGCTACCTGTATAGCGGCTACTACTTCGACTA |
| | | CTGGGGTCAAGGCACCCTGGTCACCGTGTCTAG |
| | | CGCTAGCACTAAGGGCCCCTCCGTGTTCCCTCTG |
| | | GCCCCTTCCAGCAAGTCTACCTCCGGCGGCACA |
| | | GCTGCTCTGGGCTGCCTGGTCAAGGACTACTTCC |
| | | CTGAGCCTGTGACAGTGTCCTGGAACTCTGGCG |
| | | CCCTGACCTCTGGCGTGCACACCTTCCCTGCCGT |
| | | GCTGCAGTCCTCCGGCCTGTACTCCCTGTCCTCC |
| | | GTGGTCACAGTGCCTTCAAGCAGCCTGGGCACC |
| | | CAGACCTATATCTGCAACGTGAACCACAAGCCT |
| | | TCCAACACCAAGGTGGACAAGCGGGTGGAGCCT |
| | | AAGTCCTGCGACAAGACCCACACCTGTCCTCCC |
| | | TGCCCTGCTCCTGAACTGCTGGGCGGCCCTTCTG |
| | | TGTTCCTGTTCCCTCCAAAGCCCAAGGACACCCT |
| | | GATGATCTCCCGGACCCCTGAAGTGACCTGCGT |
| | | GGTGGTGGCCGTGTCCCACGAGGATCCTGAAGT |
| | | GAAGTTCAATTGGTACGTGGACGGCGTGGAGGT |
| | | GCACAACGCCAAGACCAAGCCTCGGGAGGAAC |
| | | AGTACAACTCCACCTACCGGGTGGTGTCCGTGC |
| | | TGACCGTGCTGCACCAGGACTGGCTGAACGGCA |
| | | AAGAGTACAAGTGCAAAGTCTCCAACAAGGCCC |
| | | TGGCCGCCCCTATCGAAAAGACAATCTCCAAGG |
| | | CCAAGGGCCAGCCTAGGGAACCCCAGGTGTACA |
| | | CCCTGCCACCCAGCCGGGAGGAAATGACCAAGA |
| | | ACCAGGTGTCCCTGACCTGTCTGGTCAAGGGCTT |
| | | CTACCCTTCCGATATCGCCGTGGAGTGGGAGTCT |
| | | AACGGCCAGCCTGAGAACAACTACAAGACCACC |
| | | CCTCCTGTGCTGGACTCCGACGGCTCCTTCTTCC |
| | | TGTACTCCAAACTGACCGTGGACAAGTCCCGGT |
| | | GGCAGCAGGGCAACGTGTTCTCCTGCTCCGTGA |
| | | TGCACGAGGCCCTGCACAACCACTACACCCAGA |
| | | AGTCCCTGTCCCTGTCTCCCGGCAAG |
| LCDR1 (Kabat) | 33 | SGSSSNIGSNDVS |
| LCDR2 (Kabat) | 34 | KNYNRPS |
| LCDR3 (Kabat) | 35 | SAWDQRQFDVV |
| LCDR1 (Chothia) | 36 | SSSNIGSND |
| LCDR2 (Chothia) | 37 | KNY |
| LCDR3 (Chothia) | 38 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 39 | QSVLTQPPSASGTPGQRVTISCSGSSSNIGSNDVSW |
| | | YQQLPGTAPKLLIYKNYNRPSGVPDRFSGSKSGTS |
| | | ASLAISGLQSEDEADYYCSAWDQRQFDVVFGGGT |
| | | KLTVL |
| DNA encoding VL | 40 | CAGTCAGTCCTGACTCAGCCCCCTAGCGCTAGT |
| | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| Light Chain | 41 | QSVLTQPPSASGTPGQRVTISCSGSSSNIGSNDVSW |
| | | YQQLPGTAPKLLIYKNYNRPSGVPDRESGSKSGTS |
| | | ASLAISGLQSEDEADYYCSAWDQRQFDVVFGGGTK |
| | | LTVLGQPKAAPSVTLFPPSSEELQANKATLVCLIS |
| | | DFYPGAVTVAWKADSSPVKAGVETTTPSKQSNNK |
| | | YAASSYLSLTPEQWKSHRSYSCQVTHEGSTVEKT |
| | | VAPTECS |
| DNA encoding Light | 42 | CAGTCAGTCCTGACTCAGCCCCCTAGCGCTAGT |
| Chain | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| | | GGTCAACCTAAGGCTGCCCCCAGCGTGACCCTG |
| | | TTCCCCCCCAGCAGCGAGGAGCTGCAGGCCAAC |
| | | AAGGCCACCCTGGTGTGCCTGATCAGCGACTTC |
| | | TACCCAGGCGCCGTGACCGTGGCCTGGAAGGCC |
| | | GACAGCAGCCCCGTGAAGGCCGGCGTGGAGACC |
| | | ACCACCCCCAGCAAGCAGAGCAACAACAAGTAC |
| | | GCCGCCAGCAGCTACCTGAGCCTGACCCCCGAG |
| | | CAGTGGAAGAGCCACAGGTCCTACAGCTGCCAG |
| | | GTGACCCACGAGGGCAGCACCGTGGAAAAGAC |
| | | CGTGGCCCCAACCGAGTGCAGC |

In some embodiments, other antibodies for use in the methods or formulations described herein (e.g., methods for treating a subject afflicted with or at risk of developing a thromboembolic disorder) include those where the amino acids or nucleic acids encoding the amino acids have been mutated, yet have at least 60, 65, 70, 75, 80, 85, 90, or 95 percent identity to the sequences described in Table 1. Some embodiments include mutant amino acid sequences wherein no more than 1, 2, 3, 4 or 5 amino acids have been mutated in the variable regions when compared with the variable regions depicted in the sequence described in Table 1, while retaining substantially the same antigen binding activity.

Since each of these antibodies can bind to FXI and/or FXIa, the VH, VL, full length light chain, and full length heavy chain sequences (amino acid sequences and the nucleotide sequences encoding the amino acid sequences) can be “mixed and matched” to create other FXI and/or FXIa-binding antibodies of the present disclosure. Such “mixed and matched” FXI and/or FXIa-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

Accordingly, in one aspect, for use in the methods described herein (e.g., methods for treating a subject afflicted with or at risk of developing a thromboembolic disorder), the present disclosure provides an isolated antibody or antigen binding fragment thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa). In another aspect, for use in the formulations described herein (e.g., the formulation in the vial, the intravenous drug delivery formulation), the present disclosure provides an isolated antibody or antigen binding fragment thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa).

More specifically, in certain aspects, the present disclosure provides an isolated antibody or antigen binding fragment thereof having a heavy chain variable domain and a light chain variable domain comprising amino acid sequences selected from SEQ ID NOs: 9 and 29; or 19 and 39, respectively.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39.

In another aspect for use in the methods described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding fragment thereof having a heavy chain and a light chain comprising amino acid sequences selected from SEQ ID NOs: 11 and 31; or 21 and 41, respectively. In another aspect for use in the formulations described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding fragment thereof having a heavy chain and a light chain comprising amino acid sequences selected from SEQ ID NOs: 11 and 31; or 21 and 41, respectively.

In a specific embodiment for use in the methods described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21. In a specific embodiment for use in the formulations described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21.

In a specific embodiment for use in the methods described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41. In a specific embodiment for use in the formulations described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41.

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), Lefranc et al., (2003) Dev. Comp. Immunol., 27, 55-77 (“IMGT” numbering scheme), or the “Combined” system.

For example, under Kabat, the CDR amino acid residues of Antibody 2 in the heavy chain variable domain (VH) are numbered 31-35 (HCDR1), 50-66 (HCDR2), and 99-111 (HCDR3); and the CDR amino acid residues in the light chain variable domain (VL) are numbered 22-35 (LCDR1), 51-57 (LCDR2), and 90-100 (LCDR3). Under Chothia the CDR amino acids in the VH are numbered 26-32 (HCDR1), 52-57 (HCDR2), and 99-111 (HCDR3); and the amino acid residues in VL are numbered 25-33 (LCDR1), 51-53 (LCDR2), and 92-99 (LCDR3). By combining the CDR definitions of both Kabat and Chothia, the CDRs consist of amino acid residues 26-35 (HCDR1), 50-66 (HCDR2), and 99-111 (HCDR3) in human VH and amino acid residues 22-35 (LCDR1), 51-57 (LCDR2), and 90-100 (LCDR3) in human VL. By combining the CDR definitions of both Kabat and Chothia, the “Combined” CDRs consist of amino acid residues 26-35 (HCDR1), 50-66 (HCDR2), and 99-108 (HCDR3) in human VH and amino acid residues 24-38 (LCDR1), 54-60 (LCDR2), and 93-101 (LCDR3) in human VL. As another example, under IMGT, the CDR amino acid residues in the heavy chain variable domain (VH) are numbered 26-33 (HCDR1), 51-58 (HCDR2), and 97-108 (HCDR3); and the CDR amino acid residues in the light chain variable domain (VL) are numbered 27-36 (LCDR1), 54-56 (LCDR2), and 93-101 (LCDR3). Table 1 provides exemplary Kabat, Chothia, Combined, and IMGT HCDR1, HCDR2, HCDR3, LCDR1, LCDR2, and LCDR3 for anti-FXI/FXIa antibodies, e.g., Antibody 2 and Antibody 1. In another aspect, the present disclosure provides FXIa binding antibodies that comprise the heavy chain and light chain CDR1s, CDR2s, and CDR3s as described in Table 1, or combinations thereof. The amino acid sequences of the VH CDR1s of the antibodies are shown in SEQ ID NOs: 3 and 23. The amino acid sequences of the VH CDR2s of the antibodies are shown in SEQ ID NOs: 4 and 24. The amino acid sequences of the VH CDR3s of the antibodies are shown in SEQ ID NOs: 5 and 25. The amino acid sequences of the VL CDR1s of the antibodies are shown in SEQ ID NOs: 13 and 33. The amino acid sequences of the VL CDR2s of the antibodies are shown in SEQ ID NOs: 14 and 34. The amino acid sequences of the VL CDR3s of the antibodies are shown in SEQ ID NOs: 15 and 35. These CDR regions are delineated using the Kabat system.

Alternatively, as defined using the Chothia system (Al-Lazikani et al., (1997) JMB 273,927-948), the amino acid sequences of the VH CDR1s of the antibodies are shown in SEQ ID NOs: 6 and 26. The amino acid sequences of the VH CDR2s of the antibodies and are shown in SEQ ID NOs: 7 and 27. The amino acid sequences of the VH CDR3s of the antibodies are shown in SEQ ID NOs: 8 and 28. The amino acid sequences of the VL CDR1s of the antibodies are shown in SEQ ID NOs: 16 and 36. The amino acid sequences of the VL CDR2s of the antibodies are shown in SEQ ID NOs: 17 and 37 or have the amino acid sequence of KNY. The amino acid sequences of the VL CDR3s of the antibodies are shown in SEQ ID NOs: 18 and 38.

Alternatively, as defined using the Combined system, the amino acid sequences of the VH CDR1 of the antibodies are shown in SEQ ID NO: 46. The amino acid sequences of the VH CDR2 of the antibodies and are shown in SEQ ID NO: 4. The amino acid sequences of the VH CDR3 of the antibodies are shown in SEQ ID NO: 5. The amino acid sequences of the VL CDR1 of the antibodies are shown in SEQ ID NO: 33. The amino acid sequences of the VL CDR2 of the antibodies are shown in SEQ ID NO: 14. The amino acid sequences of the VL CDR3 of the antibodies are shown in SEQ ID NO: 15.

Alternatively, as defined using the IMGT numbering scheme, the amino acid sequences of the VH CDR1 of the antibodies are shown in SEQ ID NO: 43. The amino acid sequences of the VH CDR2 of the antibodies and are shown in SEQ ID NO: 44. The amino acid sequences of the VH CDR3 of the antibodies are shown in SEQ ID NO: 45. The amino acid sequences of the VL CDR1 of the antibodies are shown in SEQ ID NO: 47. The amino acid sequences of the VL CDR2 of the antibodies are shown in SEQ ID NO: 37 (or the amino acid sequence of KNY). The amino acid sequences of the VL CDR3 of the antibodies are shown in SEQ ID NO: 15.

Given that each of these antibodies can bind to FXI and/or FXIa and that antigen-binding specificity is provided primarily by the CDR1, 2 and 3 regions, the VH CDR1, 2 and 3 sequences and VL CDR1, 2 and 3 sequences can be “mixed and matched” (e.g., CDRs from different antibodies can be mixed and matched, although each antibody preferably contains a VH CDR1, 2 and 3 and a VL CDR1, 2 and 3 to create other FXI and/or FXIa binding molecules of the present disclosure). Such “mixed and matched” FXI and/or FXIa binding antibodies can be tested using the binding assays known in the art and those described in the Examples (e.g., ELISAs, SET, BIACORE™ assays). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure. In addition to the foregoing, in one embodiment, the antigen binding fragments of the antibodies described herein can comprise a VH CDR1, 2, and 3, or a VL CDR 1, 2, and 3, wherein the fragment binds to FXI and/or FXIa as a single variable domain. It is noted that the CDR sequences of Antibody 1 and Antibody 2 are identical.

In certain embodiments of the present disclosure, the antibodies or antigen binding fragments thereof may have the heavy and light chain sequences of the Fabs described in Table 1. More specifically, the antibody or antigen binding fragments thereof may have the heavy and light sequence of Antibody 2 and Antibody 1.

In other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Kabat and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Chothia and described in Table 1. In other embodiments, the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by the Combined system and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by IMGT and described in Table 1.

In a specific embodiment for use in the methods described herein, the present disclosure includes an antibody that specifically binds to FXI and/or FXIa comprising a heavy chain variable region CDR1 of SEQ ID NO: 3; a heavy chain variable region CDR2 of SEQ ID NO: 4; a heavy chain variable region CDR3 of SEQ ID NO: 5; a light chain variable region CDR1 of SEQ ID NO: 13; a light chain variable region CDR2 of SEQ ID NO: 14; and a light chain variable region CDR3 of SEQ ID NO: 15.

In a specific embodiment, the present disclosure includes an antibody that specifically binds to FXI and/or FXIa comprising a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35.

In a specific embodiment, the present disclosure includes an antibody that specifically binds to FXI and/or FXIa comprising a heavy chain variable region CDR1 of SEQ ID NO: 6; a heavy chain variable region CDR2 of SEQ ID NO: 7; a heavy chain variable region CDR3 of SEQ ID NO: 8; a light chain variable region CDR1 of SEQ ID NO: 16; a light chain variable region CDR2 of SEQ ID NO: 17 (or the amino acid sequence of KNY); and a light chain variable region CDR3 of SEQ ID NO: 18.

In a specific embodiment, the present disclosure includes an antibody that specifically binds to FXI and/or FXIa comprising a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37 (or the amino acid sequence of KNY); and a light chain variable region CDR3 of SEQ ID NO: 38.

In a specific embodiment, provided herein is an antibody that specifically binds to FXI and/or FXIa comprising a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37 (or the amino acid sequence of KNY) and a light chain variable region CDR3 of SEQ ID NO: 15.

In a specific embodiment, provided herein is an antibody that specifically binds to FXI and/or FXIa comprising a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14 and a light chain variable region CDR3 of SEQ ID NO: 15.

In certain embodiments, the present disclosure includes antibodies or antigen binding fragments that specifically bind to FXI and/or FXIa as described in Table 1. In a specific embodiment for use in the methods described herein, the antibody, or antigen binding fragment, that binds FXI and/or FXIa is Antibody 2 and Antibody 1.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described below, as well as DP47 and DPK9.

### Homologous Antibodies

In yet other embodiments for use in the methods described herein (e.g., methods for treating a subject afflicted with or at risk of developing a thromboembolic disorder), the present disclosure provides an antibody, or an antigen binding fragment thereof, comprising amino acid sequences that are homologous to the sequences described in Table 1 (e.g., SEQ ID NOs: 29, 31, 39, or 41), and the antibody binds to a FXI and/or FXIa protein (e.g., human, rabbit, cynomolgus monkey, and baboon FXIa), and retains the desired functional properties of those antibodies described in Table 1 such as Antibody 2 and Antibody 1. In specific aspects, such homologous antibodies retain the CDR amino acid sequences described in Table 1 (e.g., Kabat CDRs, Chothia CDRs, IMGT CDRs, or Combined CDRs).

For example, in some embodiments the present disclosure provides an isolated antibody, or a functional antigen binding fragment thereof, comprising a heavy chain variable domain and a light chain variable domain, wherein the heavy chain variable domain comprises an amino acid sequence that is at least 80%, at least 90%, or at least 95% identical to an amino acid sequence selected from the group consisting of SEQ ID NOs: 9 and 29; the light chain variable domain comprises an amino acid sequence that is at least 80%, at least 90%, or at least 95% identical to an amino acid sequence selected from the group consisting of SEQ ID NOs: 19 and 39; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 9; the light chain variable domain comprises an amino acid sequence that is at least 80%, at least 90%, or at least 95% identical to the amino acid sequence of SEQ ID NO: 19; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 29; the light chain variable domain comprises an amino acid sequence that is at least 80%, at least 90%, or at least 95% identical to the amino acid sequence of SEQ ID NO: 39; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, 17 (or the amino acid sequence of KNY), and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, 37 (or the amino acid sequence of KNY), and 15, respectively.

In other embodiments for use in the methods described herein, the VH and/or VL amino acid sequences may be 50%, 60%, 70%, 80%, 90%, 95%, 96%, 97%, 98% or 99% identical to the sequences set forth in Table 1. In other embodiments for use in the formulations described herein, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except for an amino acid substitution in no more than 1, 2, 3, 4 or 5 amino acid positions. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 10 or 30 and SEQ ID NOs: 20 and 40, respectively, followed by testing of the encoded altered antibody for retained function using the functional assays described herein.

In other embodiments for use in the methods described herein, the full length heavy chain and/or full length light chain amino acid sequences may be 50% 60%, 70%, 80%, 90%, 95%, 96%, 97%, 98% or 99% identical to the sequences set forth in Table 1 (e.g., SEQ ID NOS: 11 and/or 21, or 31 and/or 41). In other embodiments for use in the formulations described herein, the full length heavy chain and/or full length light chain amino acid sequences may be 50% 60%, 70%, 80%, 90%, 95%, 96%, 97%, 98% or 99% identical to the sequences set forth in Table 1 (e.g., SEQ ID NOs: 11 and/or 21, or 31 and/or 41). An antibody having a full length heavy chain and full length light chain having high (e.g., 80% or greater) identity to the full length heavy chains of any of SEQ ID NOs: 11 or 31, and full length light chains of any of SEQ ID NOs: 21 or 41, can be obtained by mutagenesis (e.g., site-directed or PCR-mediated mutagenesis) of nucleic acid molecules encoding such polypeptides, followed by testing of the encoded altered antibody for retained function using the functional assays described herein.

In one aspect, provided herein is an isolated antibody, or a functional antigen binding fragment thereof, comprising a heavy chain and a light chain, wherein the heavy chain comprises an amino acid sequence that is at least 80%, at least 90%, or at least 95% identical to an amino acid sequence selected from the group consisting of SEQ ID NOs: 11 and 31; the light chain comprises an amino acid sequence that is at least 80%, at least 90%, or at least 95% identical to an amino acid sequence selected from the group consisting of SEQ ID NOs: 21 and 41; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 11; the light chain comprises an amino acid sequence that is at least 80%, at least 90%, or at least 95% identical to the amino acid sequence of SEQ ID NO: 21; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 31; the light chain comprises an amino acid sequence that is at least 80%, at least 90%, or at least 95% identical to the amino acid sequence of SEQ ID NO: 41; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, 17 (or the amino acid sequence of KNY), and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, 37 (or the amino acid sequence of KNY), and 15, respectively.

In other embodiments for use in the methods described herein, the full length heavy chain and/or full length light chain nucleotide sequences may be 60%, 70%, 80%, 90%, 95%, 96%, 97%, 98% or 99% identical to the sequences set forth in Table 1 (e.g., SEQ ID NOs: 12 and/or 22, or 32 and/or 42).

In other embodiments for use in the methods described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40). In other embodiments for use in the formulations described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40).

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The isolated anti-FXI and/or FXIa antibodies, or antigen binding fragments thereof, as described herein can be monoclonal antibodies, human or humanized antibodies, chimeric antibodies, single chain antibodies, Fab fragments, Fv fragments, F(ab′) 2 fragments, or scFv fragments, and/or IgG isotypes (e.g., IgG1 such as human IgG1). In specific embodiments, anti-FXI and/or anti-FXIa antibodies described herein are recombinant human antibodies. In specific embodiments, anti-FXI and/or anti-FXIa antibodies described herein are human IgG1/lambda (2) antibodies. In specific embodiments, anti-FXI and/or anti-FXIa antibodies described herein are human IgG1/lambda (2) antibodies comprising an Fc domain engineered to reduce the potential for effector function (e.g., ADCC and/or CDC), for example a human Fc domain comprising D265A and/or P329A substitutions.

Additionally or alternatively, the protein sequences of the present disclosure can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain other embodiments, an antibody of the present disclosure for use in the methods described herein (e.g., methods for treating a subject afflicted with or at risk of developing a thromboembolic disorder) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure. In certain other embodiments, an antibody of the present disclosure for use in the formulations described herein (e.g., the formulation in the vial, the intravenous drug delivery formulation) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure.

Accordingly, for use in the methods described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

For use in the formulations described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

In one aspect, provided herein is an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of those described in Table 1, and conservative modifications thereof; the heavy chain variable region CDR2 amino acid sequences are selected from the group consisting of those described in Table 1, and conservative modifications thereof; the heavy chain variable region CDR3 amino acid sequences are selected from the group consisting of those described in Table 1, and conservative modifications thereof; the light chain variable regions CDR1 amino acid sequences are selected from the group consisting of those described in Table 1, and conservative modifications thereof; the light chain variable regions CDR2 amino acid sequences are selected from the group consisting of those described in Table 1, and conservative modifications thereof; the light chain variable regions of CDR3 amino acid sequences are selected from the group consisting of those described in Table 1, and conservative modifications thereof; and the antibody or antigen binding fragments thereof specifically binds to FXIa.

In other embodiments for use in the methods described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. In other embodiments for use in the formulations described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. Accordingly, the present disclosure provides an isolated antibody optimized for expression in a mammalian cell consisting of a full length heavy chain and a full length light chain wherein the full length heavy chain has amino acid sequences selected from the group of SEQ ID NOs: 11 or 31, and conservative modifications thereof; and the full length light chain has amino acid sequences selected from the group of SEQ ID NOs: 21 or 41, and conservative modifications thereof; and the antibody specifically binds to FXI and/or FXIa (e.g., human, rabbit, cynomolgus monkey, and baboon FXIa).

### Antibodies That Bind to the Same Epitope

In some embodiments, the present disclosure provides antibodies that compete for the same epitope as the FXI and/or FXIa binding antibodies described in Table 1, for use in the methods described herein (e.g., methods for treating a subject afflicted with or at risk of developing a thromboembolic disorder). In some embodiments, the present disclosure provides antibodies that compete for the same epitope as the FXI and/or FXIa binding antibodies described in Table 1, for use in the formulations described herein (e.g., the formulation in the vial, the intravenous drug delivery formulation). Additional antibodies can therefore be identified based on their ability to compete (e.g., to competitively inhibit the binding of, in a statistically significant manner, by binding to the same or overlapping epitope) with other antibodies of the present disclosure in FXI and/or FXIa binding assays (such as those described in the Examples Section). The ability of a test antibody to inhibit the binding of antibodies of the present disclosure to a FXI and/or FXIa protein demonstrates that the test antibody can compete with that antibody for binding to FXI and/or FXIa; such an antibody may, according to non-limiting theory, bind to the same or a related (e.g., a structurally similar or spatially proximal) epitope on the FXI and/or FXIa protein as the antibody with which it competes. In a certain embodiment, the antibody that binds to the same epitope on FXI and/or FXIa as the antibodies of the present disclosure is a human monoclonal antibody. Such human monoclonal antibodies can be prepared and isolated as described herein.

As used herein, an antibody “competes” for binding when the competing antibody binds to the same FXI and/or FXIa epitope as an antibody or antigen binding fragment of the present disclosure (e.g., Antibody 1 or Antibody 2) and inhibits FXI and/or FXIa binding of an antibody or antigen binding fragment of the present disclosure by more than 50% (for example, 80%, 85%, 90%, 95%, 98% or 99%) in the presence of an equimolar concentration of competing antibody. This may be determined, for instance, in a competitive binding assay, by any of the methods well known to those of skill in the art.

As used herein, an antibody or antigen binding fragment thereof does not “compete” with a FXI and/or FXIa antibody or antigen binding fragment of the present disclosure (e.g., Antibody 1 or Antibody 2) unless said competing antibody or antigen binding fragment thereof binds the same FXI and/or FXIa epitope, or an overlapping FXI and/or FXIa epitope, as an antibody or antigen binding fragment of the present disclosure. As used herein, a competing antibody or antigen binding fragment thereof does not include one which (i) sterically blocks an antibody or antigen binding fragment of the present disclosure from binding its target (e.g., if said competing antibody binds to a nearby, non-overlapping FXI and/or FXIa epitope and physically prevents an antibody or antigen binding fragment of the present disclosure from binding its target); and/or (ii) binds to a different, non-overlapping FXI and/or FXIa epitope and induces a conformational change to the FXI and/or FXIa protein such that said protein can no longer be bound by a FXI and/or FXIa antibody or antigen binding fragment of the present disclosure in a way that would occur absent said conformational change.

### Engineered and Modified Antibodies

In some embodiments, an antibody of the present disclosure, for use in the methods described herein, further can be prepared using an antibody having one or more of the VH and/or VL sequences shown herein as starting material to engineer a modified antibody, which modified antibody may have altered properties from the starting antibody. In some embodiments, an antibody of the present disclosure, for use in the formulations described herein, further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the present disclosure pertains to an isolated antibody, or an antigen binding fragment thereof, comprising a heavy chain variable region comprising CDR1 sequences having an amino acid sequence selected from the group consisting of SEQ ID NOs: 3 and 23; CDR2 sequences having an amino acid sequence selected from the group consisting of SEQ ID NOs: 4 and 24; CDR3 sequences having an amino acid sequence selected from the group consisting of SEQ ID NOs: 5 and 25, respectively; and a light chain variable region having CDR1 sequences having an amino acid sequence selected from the group consisting of SEQ ID NOs: 13 and 33; CDR2 sequences having an amino acid sequence selected from the group consisting of SEQ ID NOs: 14 and 34; and CDR3 sequences consisting of an amino acid sequence selected from the group consisting of SEQ ID NOs: 15 and 35, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in the antibodies of the present disclosure are those that are structurally similar to the framework sequences used by selected antibodies of the present disclosure, e.g., consensus sequences and/or framework sequences used by monoclonal antibodies of the present disclosure. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, V12, and Vk2.

Accordingly, for use in the methods described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Accordingly, for use in the formulations described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Another type of variable region modification is mutation of amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples Section. Conservative modifications (as discussed above) can be introduced. The mutations may be amino acid substitutions, additions or deletions. Moreover, typically no more than one, two, three, four or five residues within a CDR region are altered.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence selected from the group consisting of SEQ ID NOs: 17 and 37 or having the amino acid sequence of KNY, or an amino acid sequence having one, two, three, four or five amino acid substitutions, deletions or additions as compared to SEQ ID NOs: 17 and 37 or having the amino acid sequence of KNY; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence selected from the group consisting of SEQ ID NOs: 17 and 37 or having the amino acid sequence of KNY, or an amino acid sequence having one, two, three, four or five amino acid substitutions, deletions or additions as compared to SEQ ID NOs: 17 and 37 (or the amino acid sequence of KNY); and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Antibodies with Extended Half Life

In some embodiments, the present disclosure provides for antibodies that specifically bind to FXIa protein which have an extended half-life in vivo, for use in the methods or formulations described herein.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half-life of the antibodies of the present disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the present disclosure. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a nonnative amino acid any place an amber codon is positioned, converting the amber from a stop codon to one that signals incorporation of the chemically specified amino acid.

Recombinant pegylation technology (rPEG) can also be used for serum half-life extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to FXIa while a second binding domain of the antibody binds to serum albumin, preferably HSA.

The strategies for increasing half-life is especially useful in nanobodies, fibronectin-based binders, and other antibodies or proteins for which increased in vivo half-life is desired.

### Antibody Conjugates

In some embodiments, the present disclosure provides antibodies or fragments thereof, for use in the methods or formulations described herein, that specifically bind to an FXIa protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the present disclosure provides fusion proteins comprising an antigen-binding fragment of an antibody described herein (e.g., a Fab fragment, Fd fragment, Fv fragment, F (ab) 2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88:10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the present disclosure or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2): 76-82; Hansson, et al., 1999, J. Mol. Biol. 287:265-76; and Lorenzo and Blasco, 1998, Biotechniques 24 (2): 308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an FXIa protein may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide (amino acid sequence: HHHHHH; SEQ ID NO: 48), such as the tag provided in a pQE vector (QIAGEN, Inc., 9259 Eton Avenue, Chatsworth, CA, 91311), among others, many of which are commercially available. As described in Gentz et al., 1989, Proc. Natl. Acad. Sci. USA 86:821-824, for instance, hexa-histidine (SEQ ID NO: 48) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present disclosure or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and nonradioactive paramagnetic metal ions.

In some embodiments, the present disclosure further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alpha-emitters such as 213Bi or macrocyclic chelators useful for conjugating radiometal ions, including but not limited to, 131 In, 131LU, 131Y, 131Ho, 131Sm, to polypeptides. In certain embodiments, the macrocyclic chelator is 1,4,7,10-tetraazacyclododecane-N,N′,N″,N″″-tetraacetic acid (DOTA) which can be attached to the antibody via a linker molecule. Such linker molecules are commonly known in the art and described in Denardo et al., 1998, Clin Cancer Res. 4 (10): 2483-90; Peterson et al., 1999, Bioconjug. Chem. 10 (4): 553-7; and Zimmerman et al., 1999, Nucl. Med. Biol. 26 (8): 943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Formulations

In some embodiments, the present disclosure also provides pharmaceutical formulations that contain a therapeutically effective amount of a Factor XI and/or Factor XIa antibody disclosed herein (e.g., Antibody 1). The pharmaceutical formulation comprises one or more excipients and is maintained at a certain pH. Non-limiting examples of an “excipient,” as used herein, include any non-therapeutic agent added to the formulation to provide a desired physical or chemical property, for example, pH, osmolarity, viscosity, clarity, color, isotonicity, odor, sterility, stability, rate of dissolution or release, adsorption, or penetration. Exemplary pharmaceutical formulations comprising a Factor XI and/or Factor XIa antibody disclosed herein (e.g., Antibody 1) are provided, for example, in International Application Publication WO2021/127525.

### Drug Substance

Antibody 1 is a high-affinity, anti-human Factor XI monoclonal antibody. It is expressed in a Chinese hamster ovary cell line (CHO-C8TD). In some embodiments, the Antibody 1 drug substance is fully formulated for subcutaneous administration (i.e., no further excipients are added), and thus is identical in composition to the Antibody 1 drug product. In some embodiments, for intravenous administration, the Antibody 1 drug product is further diluted in an appropriate carrier. In some embodiments, the Antibody 1 drug product is diluted in a solution comprising dextrose, e.g., dextrose 5% in water (D5W).

### Excipients and pH

In some embodiments the excipients contained in the Antibody 1 drug product are pharmacopeial grade excipients. In some embodiments, the excipients in the Antibody 1 drug product comprise a histidine, a histidine salt, a sugar, and a polysorbate. In some embodiments, the excipients in the Antibody 1 drug product include L-histidine and L-histidine hydrochloride monohydrate (histidine buffer), sucrose, and polysorbate 20. Excipients may be selected for their suitability for intravenous and subcutaneous administration, providing the necessary stabilizing, buffering capacity, and tonicity. The formulation maximizes the stability of the monoclonal antibody product, and may provide a sterile solution suitable for subcutaneous or intravenous administration. In some embodiments, a sugar (e.g., sucrose) acts as a stabilizer. In some embodiments, a histidine (e.g., L-histidine, L-Histidine HCl monohydrate) acts as a buffering agent. In some embodiments, a polysorbate (e.g., polysorbate 20), acts as a stabilizer. In some embodiments, the formulation is adjusted to final volume in water for injection (WFI).

The one or more excipients in the pharmaceutical formulation of the present invention comprises a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system comprises at least one buffer that has a buffering range that overlaps fully or in part with the range of pH 5.0-7.4. In certain embodiments, the buffer has a pH of about 5.5±0.5. In certain embodiments, the buffer comprises a histidine buffer. In certain embodiments, the histidine buffer is present at a concentration of 0.05-10 mM, 0.1-10 mM, 0.2-10 mM, 0.5-10 mM, 1-10 mM, 5-10 mM, 5 to 100 mM, 10 to 100 mM, 15 to 100 mM, 20 to 100 mM, 30 to 100 mM, 40 to 100 mM, 50 to 100 mM, 60 to 100 mM, 70 to 100 mM, 80 to 100 mM, 90 to 100 mM, 5 to 90 mM, 5 to 80 mM, 5 to 70 mM, 5 to 60 mM, 5 to 50 mM, 5 to 40 mM, 5 to 30 mM, 5 to 20 mM, 10 to 50 mM, 10 to 40 mM, 10 to 30 mM, 10 to 20 mM, 5 to 25 mM, 10 to 25 mM, 15 to 25 mM, 20 to 25 mM, 5 to 20 mM, 10 to 20 mM, or 15 to 20 mM. In certain embodiments, the histidine is present at a concentration of about 0.1 mM, 0.2 mM, 0.5 mM, 1 mM, 5 mM, about 10 mM, about 15 mM, about 20 mM, about 25 mM, or about 50 mM. In certain embodiments, the histidine buffer is present at a concentration of about 20 mM. In certain embodiments, the histidine buffer is present at a concentration of about 0.20 mM. In certain embodiments, the histidine buffer has a pH of about 5.0, about 5.5, about 6.0, about 6.5, or about 7.0. In a particular embodiment, the histidine buffer has a pH of about 5.5.

The pharmaceutical formulation of the present invention may have a pH of 5.0 to 6.0. For example, in certain embodiments, the pharmaceutical formulation has a pH of 5.0 to 6.0 (i.e., 5.5±0.5), 5.1 to 5.9 (i.e., 5.5±0.4), 5.2 to 5.8 (i.e., 5.5±0.3), 5.3 to 5.7 (i.e., 5.5±0.2), 5.4 to 5.6 (i.e., 5.5±0.1), or 5.45 to 5.55 (i.e., 5.5±0.05). In certain embodiments, the pharmaceutical formulation has a pH of about 5.5, about 5.6, about 5.7, about 5.8, about 5.9, about 6.0, about 6.1, about 6.2, about 6.3, about 6.4, or about 6.5. In certain embodiments, the pharmaceutical formulation has a pH of about 5.5. Under the rules of scientific rounding, a pH greater than or equal to 5.45 and smaller than or equal to 5.55 is rounded as 5.5.

In certain embodiments, the buffer system of the pharmaceutical formulation comprises histidine at 10 to 30 mM, at a pH of 5.5±0.2. In certain embodiments, the buffer system of the pharmaceutical formulation comprises histidine at about 20 mM, at a pH of 5.5±0.2. In certain embodiments, the buffer system of the pharmaceutical formulation comprises histidine at 10 to 30 mM, at a pH of 5.5±0.05. In certain embodiments, the buffer system of the pharmaceutical formulation comprises histidine at about 20 mM, at a pH of 5.5±0.05.

In certain embodiments, the buffer system of the pharmaceutical formulation comprises histidine at 0.10 to 0.30 mM, at a pH of 5.5±0.2. In certain embodiments, the buffer system of the pharmaceutical formulation comprises histidine at about 0.20 mM, at a pH of 5.5±0.2. In certain embodiments, the buffer system of the pharmaceutical formulation comprises histidine at 0.10 to 0.30 mM, at a pH of 5.5±0.05. In certain embodiments, the buffer system of the pharmaceutical formulation comprises histidine at about 0.20 mM, at a pH of 5.5±0.05.

The one or more excipients in the pharmaceutical formulation of the present invention further comprises a sugar or sugar alcohol. Sugars and sugar alcohols are useful in pharmaceutical formulations as a thermal stabilizer. In certain embodiments, the pharmaceutical formulation comprises a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation comprises sucrose. In certain embodiments, the pharmaceutical composition comprises a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In specific embodiments, the pharmaceutical formulation comprises sucrose.

The amount of the sugar or sugar alcohol contained within the formulation can vary depending on the specific circumstances and intended purposes for which the formulation is used. In certain embodiments, the pharmaceutical formulation comprises 50 to 300 mM, 50 to 250 mM, 100 to 300 mM, 100 to 250 mM, 150 to 300 mM, 150 to 250 mM, 200 to 300 mM, 200 to 250 mM, or 250 to 300 mM of the sugar or sugar alcohol. In certain embodiments, the pharmaceutical formulation comprises about 50 mM, about 75 mM, about 100 mM, about 125 mM, about 150 mM, about 200 mM, about 220 mM, about 250 mM, or about 300 mM of the sugar or sugar alcohol. In specific embodiments, the pharmaceutical formulation comprises about 220 mM of the sugar or sugar alcohol (e.g., sucrose).

The amount of the sugar or sugar alcohol contained within the formulation can vary depending on the specific circumstances and intended purposes for which the formulation is used. In certain embodiments, the pharmaceutical formulation comprises 0.50 to 3.00 mM, 0.50 to 2.50 mM, 1.00 to 3.00 mM, 1.00 to 2.50 mM, 1.50 to 3.00 mM, 1.50 to 2.50 mM, 2.00 to 3.00 mM, 2.00 to 2.50 mM, or 2.50 to 3.00 mM of the sugar or sugar alcohol. In certain embodiments, the pharmaceutical formulation comprises about 0.50 mM, about 0.75 mM, about 1.00 mM, about 1.25 mM, about 1.50 mM, about 2.00 mM, about 2.20 mM, about 2.50 mM, or about 3.00 mM of the sugar or sugar alcohol. In specific embodiments, the pharmaceutical formulation comprises about 2.20 mM of the sugar or sugar alcohol (e.g., sucrose).

The one or more excipients in the pharmaceutical formulation disclosed herein further comprises a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 20.

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.02% to 0.06%, 0.03% to 0.05%, or 0.035% to 0.045% of the non-ionic surfactant (e.g., polysorbate 20). In certain embodiments, the pharmaceutical formulation comprises about 0.005%, about 0.01%, about 0.02%, about 0.03%, about 0.04%, about 0.05%, about 0.06%, about 0.07%, about 0.08%, about 0.09%, or about 0.1% of the non-ionic surfactant (e.g., polysorbate 20).

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.0002% to 0.0006%, 0.0003% to 0.0005%, or 0.00035% to 0.00045% of the non-ionic surfactant (e.g., polysorbate 20). In certain embodiments, the pharmaceutical formulation comprises about 0.00005%, about 0.0001%, about 0.0002%, about 0.0003%, about 0.0004%, about 0.0005%, about 0.0006%, about 0.0007%, about 0.0008%, about 0.0009%, or about 0.001% of the non-ionic surfactant (e.g., polysorbate 20).

In certain embodiments, the drug product is diluted in an aqueous carrier suitable for the route of administration, e.g., intravenous administration. Exemplary carriers include sterile water for injection (SWFI), bacteriostatic water for injection (BWFI), a pH buffered solution (e.g., phosphate-buffered saline), sterile saline solution, Ringer's solution, or dextrose solution. In one embodiment, when the pharmaceutical formulation is prepared for intravenous administration, the pharmaceutical formulation can be diluted in a 5% dextrose solution (D5W).

### Exemplary Formulations

In certain embodiments, the pharmaceutical formulation of the present invention comprises an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), histidine buffer, a sugar or sugar alcohol (e.g., sucrose), and a polysorbate (e.g., polysorbate 20), at pH 5.5 to 6.5.

In certain embodiments, the pharmaceutical formulation comprises 100 to 200 mg/mL of an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of a sugar or sugar alcohol (e.g., sucrose), and 0.02% to 0.06% of a polysorbate (e.g., polysorbate 20), at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.0 to 6.0. In certain embodiments, the pharmaceutical formulation comprises 100 to 200 mg/mL of the Factor XI and/or Factor XIa antibody, about 20 mM of histidine buffer, about 220 mM of a sugar or sugar alcohol (e.g., sucrose), and about 0.04% of a polysorbate (e.g., polysorbate 20), at pH 5.2 to 5.8. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of a sugar or sugar alcohol (e.g., sucrose), and 0.0002% to 0.0006% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.2 to 5.8. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.45 to 5.55.

In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of sucrose, and 0.02% to 0.06% of polysorbate 20, at pH 5.0 to 6.0. In certain embodiments, the pharmaceutical formulation comprises 100 to 200 mg/mL of the Factor XI and/or Factor XIa antibody, about 20 mM of histidine buffer, about 220 mM of sucrose, and about 0.04% of polysorbate 20, at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.3 to 5.7. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of sucrose, and 0.0002% to 0.0006% of polysorbate 20, at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.0 to 6.0. In certain embodiments, the pharmaceutical formulation comprises 1.00 to 2.00 mg/mL of the Factor XI and/or Factor XIa antibody, 20 mM of histidine buffer, about 2.20 mM of sucrose, and about 0.0004% of polysorbate 20, at pH 5.3 to 5.7. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.45 to 5.55.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

Stability of the Factor XI and/or Factor XIa Antibody

The pharmaceutical formulations of the present invention exhibit high levels of stability. A pharmaceutical formulation is stable when the Factor XI and/or Factor XIa antibody within the formulation retains an acceptable degree of physical property, chemical structure, and/or biological function after storage under defined conditions.

Exemplary methods to determine stability of the Factor XI and/or Factor XIa antibody in the pharmaceutical formulation are described in Example 1 of the present disclosure. Additionally, stability of the protein can be assessed by measuring the binding affinity of the Factor XI and/or Factor XIa antibody to its targets or the biological activity of the Factor XI and/or Factor XIa antibody in certain in vitro assays, such as the aPTT and FXI activity assays described in WO 2016/207858.

The pharmaceutical formulation can be prepared and stored as a liquid formulation. In certain embodiments, the pharmaceutical formulation is a liquid formulation for storage at 2-8° C. (e.g., 4° C.). In certain embodiments, the pharmaceutical formulation is a liquid formulation for storage at 4° C. and protected from light.

Stability studies have found Antibody 1 150 mg/mL concentrate for solution for injection to be compatible with its excipients and primary packaging materials. Antibody 1 150 mg/mL concentrate for injection is suitable for subcutaneous administration with disposable syringes, without dilution or with dilution in a carrier buffer, e.g., 5% dextrose (D5W). Concentrate for injection with commercially available disposable syringes has been demonstrated for a dose range from 0.5 mg/subject to 600 mg/subject. Materials found to be compatible with Antibody 1 comprise injection syringes composed of polypropylene or polycarbonate, and needles for injection composed of stainless steel. Compatibility of Antibody 1 concentrate for solution for injection has been demonstrated with 1 mL syringes for Antibody 1 concentrations from 0.5 mg/mL to 150 mg/mL. Compatibility of Antibody 1 concentrate for solution for injection has been demonstrated with 3 mL syringes filled up to approximately 2 mL for an Antibody 1 concentration of 150 mg/mL, covering in total a dose range from 0.5 mg up to 150 mg for the 1 mL syringe and a dose of about 300 mg for the 3 mL syringe (filled with approximately 2 mL) per injection.

### Dosage Forms

Prior to pharmaceutical use, the pharmaceutical formulation can be diluted in an aqueous carrier if suitable for the route of administration. For intravenous administration, suitable carriers include sterile water for injection (SWFI), bacteriostatic water for injection (BWFI), a pH buffered solution (e.g., phosphate-buffered saline), sterile saline solution, Ringer's solution, or dextrose solution. For example, when the pharmaceutical formulation is prepared for intravenous administration, the pharmaceutical formulation comprises a 5% dextrose solution (D5W). In certain embodiments, the diluted pharmaceutical formulation is isotonic and suitable for administration by intravenous infusion, e.g., D5W. In certain embodiments, the formulation is diluted in about 50 mL D5W, 100 mL D5W, 150 mL D5W, 200 mL D5W, 250 mL D5W, 300 mL D5W, 350 mL D5W, 400 mL D5W, 450 mL D5W, 500 mL D5W, or 1 L D5W.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 100-200 mg/mL, 100-190 mg/mL, 100-180 mg/mL, 100-170 mg/mL, 100-160 mg/mL, 110-150 mg/mL, 120-150 mg/mL, 130-150 mg/mL, 140-150 mg/mL, 140-160 mg/mL, 140-170 mg/mL, 140-180 mg/mL, 140-190 mg/mL, 150-190 mg/mL, 150-180 mg/mL, 150-170 mg/mL, or 150-160 mg/mL. In certain embodiments, the pharmaceutical formulation comprises the Factor XI and/or Factor XIa antibody at a concentration of about 10 mg/mL, about 15 mg/mL, about 25 mg/mL, about 50 mg/mL, about 75 mg/mL, about 100 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, or about 200 mg/mL.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 1.00-2.00 mg/mL, 1.00-1.90 mg/mL, 1.00-1.80 mg/mL, 1.00-1.70 mg/mL, 1.00-1.60 mg/mL, 1.10-1.50 mg/mL, 1.20-1.50 mg/mL, 1.30-1.50 mg/mL, 1.40-1.50 mg/mL, 1.40-1.60 mg/mL, 1.40-1.70 mg/mL, 1.40-1.80 mg/mL, 1.40-1.90 mg/mL, 1.50-1.90 mg/mL, 1.50-1.80 mg/mL, 1.50-1.70 mg/mL, or 1.50-1.60 mg/mL. In certain embodiments, the pharmaceutical formulation comprises the Factor XI and/or Factor XIa antibody at a concentration of about 0.10 mg/mL, about 0.15 mg/mL, about 0.25 mg/mL, about 0.50 mg/mL, about 0.75 mg/mL, about 1.00 mg/mL, about 1.20 mg/mL, about 1.25 mg/mL, about 1.30 mg/mL, about 1.35 mg/mL, about 1.40 mg/mL, about 1.45 mg/mL, about 1.50 mg/mL, about 1.55 mg/mL, about 1.60 mg/mL, about 1.65 mg/mL, about 1.70 mg/mL, about 1.75 mg/mL, about 1.80 mg/mL, about 1.85 mg/mL, about 1.90 mg/mL, about 1.95 mg/mL, or about 2.00 mg/mL.

In certain embodiments, the pharmaceutical formulation is packaged in a vial (e.g., a vial, bag, pen, or syringe). In certain embodiments, the vial comprises an overfill to allow for complete removal of the intended dose. In certain embodiments, the vial comprises an overfill of 5 to 35%, 10 to 30%, 15 to 25%, or 10 to 20%. In a particular embodiment, the vial comprises an overfill of about 20%.

In certain embodiments, the formulation may be a liquid formulation. In certain embodiments, the amount of Factor XI and/or Factor XIa antibody in the container is suitable for administration as a single dose. In certain embodiments, the amount of Factor XI and/or Factor XIa antibody in the container is suitable for administration in multiple doses. In certain embodiments, the pharmaceutical formulation comprises the Factor XI and/or Factor XIa antibody at an amount of 0.1 to 200 mg. In certain embodiments, the pharmaceutical formulation comprises the Factor XI and/or Factor XIa antibody at an amount of 1 to 200 mg, 10 to 200 mg, 20 to 200 mg, 50 to 200 mg, 100 to 200 mg, 200 to 200 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation comprises the Factor XI and/or Factor XIa antibody at an amount of about 0.1 mg, about 0.5 mg, about 1 mg, about 1.5 mg, about 2 mg, about 2.5 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 450 mg, about 500 mg, about 600 mg, about 700 mg, about 800 mg, about 900 mg, about 1000 mg, about 1500 mg, or about 2000 mg in the therapeutically effective amount.

### Dosage Regimens and Therapeutic Uses

In another aspect, the present disclosure provides a method for treating thromboembolic disease, the method comprising administering to a subject in need thereof a Factor XI and/or Factor XIa antibody disclosed herein (e.g., Antibody 1) once a month.

In certain embodiments, the method further comprises administering to the subject, after the initial treatment cycle, the Factor XI and/or Factor XIa antibody in one or more monthly treatment cycles, e.g., for a period of 3-months, wherein the Factor XI and/or Factor XIa antibody is administered on Day 1, Day 31, and Day 61. The subsequent treatment cycles, in which the subject receives administration of the Factor XI and/or Factor XIa antibody once month, are designed to maintain a certain level of the Factor XI and/or Factor XIa antibody in the subject. In certain embodiments, the subject receives at least 1, 2, 3, 4, 5, 6, 7, 8, 9, 10, 11, 12, 13, 14, or 15 subsequent treatment cycles. In some embodiments, the subject remains on the treatment for life.

In some embodiments, the method comprises treating a disease or disorder in a subject in need thereof, e.g., a thromboembolic disease, comprising administering a first dose of an anti-FXI/FXIa antibody or antigen-binding fragment thereof (e.g., Antibody 1), wherein the first dose is administered intravenously, and administering a second dose of an anti-FXI/FXIa antibody or antigen-binding fragment thereof (e.g., Antibody 1), wherein the second dose is administered subcutaneously. In some embodiments, the method further comprises administering a third dose subcutaneously. In some embodiments, the method further comprises administering a fourth dose subcutaneously. In some embodiments, the method further comprises administering a fifth dose subcutaneously. In some embodiments, the method further comprises administering a sixth dose subcutaneously. In some embodiments, the method further comprises administering a seventh dose subcutaneously. In some embodiments, the method further comprises administering an eighth dose subcutaneously. In some embodiments, the method further comprises administering a ninth dose subcutaneously. In some embodiments, the method further comprises administering a tenth dose subcutaneously. In some embodiments, the method further comprises administering an eleventh dose subcutaneously. In certain embodiments, the method comprises administering a first dose intravenously, and five subsequent doses subcutaneously. In certain embodiments, the treatment duration is about six months. In certain embodiments, the method comprises administering a first dose intravenously, and eleven subsequent doses subcutaneously. In certain embodiments, the treatment duration is about a year. In certain embodiments, the method comprises administering a first dose intravenously, and subsequently administering monthly subcutaneous doses, until resolution of the disease or disorder in the subject, or for the subject's lifetime.

In some embodiments, the subject afflicted with or at risk of developing a thromboembolic disorder and who is undergoing a surgical procedure is administered the intravenous drug delivery formulation on the same day as the surgical procedure. In some embodiments, the intravenous drug delivery formulation is administered between 2 to 10 hours after surgery. In some embodiments, the intravenous drug delivery formulation is administered between 4 to 8 hours after surgery. In some embodiments, the intravenous drug delivery formulation is administered about 1 hour, about 2 hours, about 3 hours, about 4 hours, about 5 hours, about 6 hours, about 7 hours, about 8 hours, about 9 hours, or about 10 hours after surgery.

In certain embodiments, the one or more doses in the initial and subsequent treatment cycles comprise the Factor XI and/or Factor XIa antibody administered subcutaneously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered subcutaneously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, or about 200 mg. In some embodiments, the Factor XI and/or Factor XIa antibody is administered subcutaneously at a dose of about 90 mg. In some embodiments, the Factor XI and/or Factor XIa antibody is administered subcutaneously at a dose of about 120 mg. In some embodiments, the Factor XI and/or Factor XIa antibody is administered subcutaneously at a dose of about 150 mg. In some embodiments, the Factor XI and/or Factor XIa antibody is administered subcutaneously at a dose of about 180 mg. In any of the above embodiments, the Factor XI and/or Factor XIa antibody is administered subcutaneously monthly.

In some embodiments, the therapeutically effective dose range for the Factor XI and/or Factor XIa antibody (e.g., Antibody 1) following subcutaneous administration is about 75 mg to about 165 mg, about 80 mg to about 160 mg, about 85 mg to about 155 mg, or about 90 mg to about 160 mg. In certain embodiments, the therapeutically effective dose range for the Factor XI and/or Factor XIa antibody (e.g., Antibody 1) following subcutaneous administration is about 90 mg to about 160 mg.

In certain embodiments, the one or more doses in the initial and subsequent treatment cycles comprise the Factor XI and/or Factor XIa antibody (e.g., Antibody 1) are administered intravenously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered intravenously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, about 200 mg, about 250 mg, about 500 mg, about 750 mg, about 1000 mg, about 1250 mg, about 1500 mg, or about 2000 mg. In some embodiments, the Factor XI and/or Factor XIa antibody is administered intravenously at a dose of about 30 mg. In some embodiments, the Factor XI and/or Factor XIa antibody is administered intravenously at a dose of about 60 mg. In some embodiments, the Factor XI and/or Factor XIa antibody is administered intravenously at a dose of about 75 mg. In some embodiments, the Factor XI and/or Factor XIa antibody is administered intravenously at a dose of about 150 mg. In some embodiments, the Factor XI and/or Factor XIa antibody is administered intravenously in a single dose. In some embodiments, the FXI/FXIa antibody (e.g., Antibody 1) is administered in a single dose of about 150 mg. In some embodiments, the FXI/FXIa antibody (e.g., Antibody 1) is administered in a single dose of about 1000 mg. In some embodiments, the FXI/FXIa antibody (e.g., Antibody 1) is administered in a single dose of about 1500 mg. In some embodiments, the FXI/FXIa antibody (e.g., Antibody 1) is administered in a single dose of about 2000 mg.

In some embodiments, a first dose of a FXI/FXIa antibody (e.g., Antibody 1) is administered at one dose, and a second dose of the FXI/FXIa antibody (e.g., Antibody 1) is administered at a second dose. In certain embodiments, the first and second dose are the same (e.g., 150 mg). In certain embodiments, the first dose is higher than the second dose (e.g., the first dose is about 1000 mg, and the second dose is 150 mg). In certain embodiments, the method further comprises administration of subsequent doses at the same dosage as the second dose.

In certain embodiments, administering a single dose of a FXI/FXIa antibody (e.g., Antibody 1) prolongs activated partial thromboplastin time (aPTT) by 400 hours or more, e.g., 400 hours or more, 500 hours or more, or 600 hours or more. In certain embodiments, administering a single dose of a FXI/FXIa antibody (e.g., Antibody 1) prolongs activated partial thromboplastin time (aPTT) by about 600 hours.

A physician can start doses of the antibodies of the present disclosure (e.g., Antibody 1) employed in the pharmaceutical composition at levels lower than that required to achieve the desired therapeutic effect and gradually increase the dosage until the desired effect is achieved. In general, effective doses of the compositions of the present disclosure, for the treatment of thromboembolic disorders described herein vary depending upon many different factors, including means of administration, target site, physiological state of the patient, other medications administered, and whether treatment is prophylactic or therapeutic. Treatment dosages may be titrated to optimize safety and efficacy. For systemic administration with an antibody, the dosage ranges from about 0.01 to 15 mg/kg of the host body weight. For administration (e.g., subcutaneous or intravenous administration) with an antibody, the dosage may range from 0.1 mg to 5 mg or from 1 mg to 600 mg. For example, an anti-FXI/FXIa antibody described herein (e.g., Antibody 1) can be administered at a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the Factor XI and/or Factor XIa antibody is administered intravenously. For example, in certain embodiments, the Factor XI and/or Factor XIa antibody is administered by intravenous infusion, e.g., with a prefilled bag, a prefilled pen, or a prefilled syringe. In certain embodiments, the Factor XI and/or Factor XIa antibody, in a pharmaceutical formulation disclosed herein, is diluted prior to administration. For example, in certain embodiments, the pharmaceutical formulation is diluted with dextrose 5% in water (D5W) and is administered intravenously from a bag. The intravenous infusion may be for about one hour (e.g., 50 to 80 minutes). In certain embodiments, the bag is connected to a channel comprising a tube and/or a needle.

In certain embodiments, the Factor XI and/or Factor XIa antibody is administered parenterally. In certain embodiments, the Factor XI and/or Factor XIa antibody is administered parenterally in one or more doses.

The types of thromboembolic disorders that can be treated with the Factor XI and/or Factor XIa antibody or pharmaceutical formulation disclosed herein include but are not limited to a “thromboembolic,” or similar terms as used herein, can also refer to any number of the following, which the anti-FXI and/or FXIa antibodies or antigen binding fragments thereof of the present disclosure can be used to prevent or treat: thromboembolism in subjects with suspected or confirmed cardiac arrhythmia such as paroxysmal, persistent or permanent atrial fibrillation or atrial flutter; stroke prevention in atrial fibrillation (SPAF), a subpopulation of which is AF patients undergoing percutaneous coronary interventions (PCI); acute venous thromboembolic events (VTE) treatment and extended secondary VTE prevention in patients at high risk for bleeding; cerebral and cardiovascular events in secondary prevention after transient ischemic attack (TIA) or non-disabling stroke and prevention of thromboembolic events in heart failure with sinus rhythm; venous thromboembolism in pediatric subjects (pediatric VTE); clot formation in left atrium and thromboembolism in subjects undergoing cardioversion for cardiac arrhythmia; thrombosis before, during and after ablation procedure for cardiac arrhythmia; venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins; thrombosis on any artificial surface in the veins or arteries like catheter, pacemaker wires, synthetic arterial grafts; arterio-venous (AV) shunt; mechanical or biological heart valves or left ventricular assist device; pulmonary embolism in patients with or without venous thrombosis; Chronic Thromboembolic Pulmonary Hypertension (CTEPH); arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension; thrombosis and thromboembolism in patients undergoing percutaneous coronary interventions (PCI); cardioembolic and cryptogenic strokes; non-central nervous systemic embolism (non-CNS systemic embolism); hemorrhagic stroke; thrombosis in patients with invasive and non-invasive cancer malignancies (e.g., gastrointestinal cancer and genitourinary cancer); thrombosis over an indwelling catheter; thrombosis and thromboembolism in severely ill patients; cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart; thromboembolism in patients with valvular heart disease with or without atrial fibrillation; thromboembolism over valvular mechanic or biologic prostheses; thromboembolism in patients who had native or artificial cardiac patches, arterial or venous conduit tubes after heart repair of simple or complex cardiac malformations; venous thrombosis and thromboembolism after knee replacement surgery, hip replacement surgery, and orthopedic surgery, thoracic or abdominal surgery; arterial or venous thrombosis after neurosurgery including intracranial and spinal cord interventions; congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia; thrombosis and thromboembolism in chronic kidney disease; and thrombosis and thromboembolism in patients undergoing hemodialysis and in patients undergoing extra-corporal membrane oxygenation. In certain embodiments, the subject treated with the Factor XI and/or Factor XIa antibody or pharmaceutical formulation disclosed herein is obese (e.g., severely obese, e.g., with body-mass index (BMI)≥35 kg/m²). In certain embodiments, the subject treated with the Factor XI and/or Factor XIa antibody or pharmaceutical formulation disclosed herein is not obese. In certain embodiments, the obese subject is associated with lower exposure following administration of the same dose of the Factor XI and/or Factor XIa antibody (e.g., Antibody 1), as the non-obese subject. In certain embodiments, the exposure is about 10%, about 20%, about 30%, about 40%, about 50%, about 60%, about 70%, about 80%, or about 90% less for the obese subject following administration of the same dose of the Factor XI and/or Factor XIa antibody (e.g., Antibody 1), as the non-obese subject. In certain embodiments, the obese subject is associated with shorter duration of aPTT prolongation following administration of the same dose of the Factor XI and/or Factor XIa antibody (e.g., Antibody 1), as the non-obese subject. In certain embodiments, the aPTT prolongation is about 10%, about 20%, about 30%, about 40%, about 50%, about 60%, about 70%, about 80%, or about 90% shorter for the obese subject following administration of the same dose of the Factor XI and/or Factor XIa antibody (e.g., Antibody 1), as the non-obese subject.

In certain embodiments, the Factor XI and/or Factor XIa antibody (e.g., Antibody 1), is administered to a subject with a cancer. It is estimated that venous thromboembolism (VTE) affects between 1% and 8% of all cancer patients, and further, is the second leading cause of death in outpatients undergoing chemotherapy. Treatment of such VTE in cancer patients is complicated by thrombocytopenia, which is widely prevalent in cancer patients undergoing chemotherapy (Bannow et al. (2019). Res. Pract. Throm. Haemost., 2:664-669).

In certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the cancer is selected from the group consisting of gastrointestinal cancer and genitourinary cancer.

In some embodiments, the anti-FXI/FXIa antibody or antigen-binding fragment described herein (e.g., Antibody 1) is administered preventively, e.g., to prevent a clotting in a subject at risk of thrombosis. In some embodiments, the anti-FXI/FXIa antibody or antigen-binding fragment described herein (e.g., Antibody 1) is administered therapeutically, e.g., to treat a clot in a subject at risk of thrombosis.

In some embodiments, the anti-FXI/FXIa antibody or antigen-binding fragment thereof (e.g., Antibody 1) is administered to a subject with a cancer-associated thrombosis (CAT). In certain embodiments, the subject with the cancer-associated thrombosis has an existing clot. In certain embodiments, the subject has a microclot. In certain embodiments, the subject has microthombosis associated with CAT.

The CHA2DS2-VASc risk score is a validated and widely used stratification tool to predict thromboembolic risk in AF patients and to identify patients who should benefit from anticoagulation therapy (LIP 2011; Camm, et al. (2012) Eur Heart J 2012; 33:2719-2747); the accumulated evidence shows that CHA2DS2-VASc is at least as accurate as or possibly better than, scores such as CHADS2 in identifying patients who develop stroke and thromboembolism and definitively better at identifying ‘truly low-risk’ patients with AF. The CHA2DS2-VASc risk score ranges from 0 to a maximum score of 9. In certain embodiments, the subject treated with the Factor XI and/or Factor XIa antibody or pharmaceutical formulation disclosed herein has a CHA2DS2-VASc risk score of 0-1 for men and 1-2 for women. In certain embodiments, the subject treated with the Factor XI and/or Factor XIa antibody or pharmaceutical formulation disclosed herein has a CHA2DS2-VASc risk score ≥2 for men and ≥3 for women. In certain embodiments, the subject treated with the Factor XI and/or Factor XIa antibody or pharmaceutical formulation disclosed herein has a CHA2DS2-VASc risk score ≥4 or ≥3 with at least 1 of planned concomitant use of anti-platelet medication (e.g., aspirin and/or P2Y12 inhibitor) or CrCl≤50 ml/min by the Cockcroft-Gault equation.

The Factor XI and/or Factor XIa antibody disclosed herein (e.g., Antibody 1) can be used as a monotherapy or in combination with one or more therapies. Such combination therapies may be useful for treating thromboembolic disorders, such as, ischemic stroke (cardioembolic, thrombotic) or systemic embolism, AF, stroke prevention in AF (SPAF), deep vein thrombosis, venous thromboembolism, pulmonary embolism, acute coronary syndromes (ACS), acute limb ischemia, chronic thromboembolic pulmonary hypertension, or systemic embolism). In certain embodiments, the Factor XI and/or Factor XIa antibody is used as a monotherapy in accordance with the dosage regimen disclosed herein. In other embodiments, the Factor XI and/or Factor XIa antibody is used in combination with one or more therapies, wherein the Factor XI and/or Factor XIa antibody is administered in accordance with the dosage regimen disclosed herein and the one or more therapies are administered in accordance with a dosage regimen known to be suitable for treating the particular subject with the particular disorder.

In some aspects, statin therapies may be used in combination with the FXI/FXIa antibodies and antigen binding fragments, or formulations comprising said FXI/FXIa antibodies and antigen binding fragments (e.g., Antibody 1), described in the present disclosure for the treatment of patients with thrombotic and/or thromboembolic disorders. In particular aspects, non-limiting examples of therapeutic active agents suitable for use in combination with an anti-FXI/FXIa antibody described herein (e.g., Antibody 1) include thromboxane inhibitors (e.g., aspirin), adenosine diphosphate receptor antagonists (or P2Y12 inhibitors) such as thienopyridines (e.g., clopidogrel and prasugrel) and nonthienopyridines (e.g., ticagrelor and cangrelor), protease-activated receptor-1 (PAR1) antagonists (e.g., vorapaxar and atopaxar), and proton pump inhibitors (PPIs) (e.g., omeprazole, diazepam, phenytoin, lansoprazole, dexlansoprazole, rabeprazole, pantoprazole, esomeprazole, and naproxen). The use of PPIs in combination therapy may be suitable in cases where a subject has or has a history of a GI disorder, such as previous GI bleed or antecedent of peptic ulcer. In one aspect, the subject is being treated with non-steroidal anti-inflammatory drugs (NSAIDs), and is administered an anti-FXI/FXIa antibody described herein (e.g., Antibody 1) in combination with a proton pump inhibitor (e.g., omeprazole, diazepam, phenytoin, lansoprazole, dexlansoprazole, rabeprazole, pantoprazole, esomeprazole, and naproxen). In certain embodiments, a subject treated with the FXI/FXIa antibodies and antigen binding fragments, or formulations comprising said FXI/FXIa antibodies and antigen binding fragments (e.g., Antibody 1), are administered a direct oral anticoagulant (DOAC) following the duration of treatment (e.g., on the same day as end of treatment). In certain embodiments, a subject treated with the FXI/FXIa antibodies and antigen binding fragments, or formulations comprising said FXI/FXIa antibodies and antigen binding fragments (e.g., Antibody 1), are administered a Vitamin K Antagonist (VKA) following the duration of treatment (e.g., about 5 days before end of treatment, or about 3 days before end of treatment).

In certain embodiments, the method of treatment disclosed herein results in a disease response or improved survival of the subject or patient. For example, in certain embodiments, the disease response is a complete response, a partial response, or a stable disease. In certain embodiments, the improved survival is improved progression-free survival (PFS) or overall survival. Improvement (e.g., in PFS) can be determined relative to a period prior to initiation of the treatment of the present disclosure. Methods of determining disease response (e.g., complete response, partial response, or stable disease) and patient survival (e.g., PFS, overall survival) for BTC (e.g., advanced BTC, metastatic BTC), or biliary tract tumor therapy, are routine in the art and are contemplated herein. In some embodiments, disease response is evaluated according to RECIST 1.1 after subjecting the treated patient to contrast-enhanced computed tomography (CT) or magnetic resonance imaging (MRI) of the affected area (e.g., chest/abdomen and pelvis covering the area from the superior extent of the thoracic inlet to the symphysis pubis).

### Combination Therapies and Related Dosage Regimens

### Combination Therapies for Reversal of Anti-Coagulant Effects

In one aspect, provided herein is a method of reversing an anticoagulant effect of an isolated anti-Factor XI (FXI) and/or anti-activated Factor XI (FXIa) antibody, or an antigen-binding fragment thereof, in a subject being administered the isolated antibody or antigen-binding fragment thereof at a dose of about 150 mg, wherein the method comprises administering to the subject a therapeutically effective amount of recombinant activated factor VII (rFVIIa), thereby reversing the anticoagulant effect. In certain embodiments, such a method is used, e.g., for a subject at high risk of bleeding.

In some embodiments, a temporary reversal or inhibition of one or more of the anticoagulant effects of an anti-FXI/FXIa antibody or antigen-binding fragment described herein (e.g., Antibody 1) is desired and/or medically necessary, e.g., for a subject at high risk of bleeding. In some embodiments, bleeding is typically associated with, for example and without limitation, trauma, surgery, menstruation, or postpartum. Therefore, under these circumstances, a subject, who has been treated with an anti-FXI/FXIa antibody or antigen-binding fragment described herein (e.g., Antibody 1) may be in need of quick and effective therapy, to reduce bleeding or to reduce bleeding risk. In certain embodiments, prolonged bleeding may occur after a major trauma or after surgery, for example and without limitation, surgery involving organs with a high fibrinolytic area such as buccal, nasal, genital, or urinary mucosa. Tooth extraction, tonsillectomy, and ablation of the uterus or prostate are further non-limiting examples of surgeries that entail a high risk of bleeding. In certain embodiments, concomitant use of antiplatelets, other anticoagulants, and fibrinolytic agents can increase the risk of bleeding. In some embodiments, a subject at high risk of bleeding may be identified by previous medical history of bleeding, for example, bleeding during or after surgery, or bleeding when treated with an anticoagulant (e.g. warfarin). In some embodiments, a subject at high risk of bleeding, may be identified by in vitrolex vivo assays known in the art, for example and without limitation, assays with a subject's blood or plasma: measuring aPTT, assessing biomarkers of the extrinsic coagulation pathways, measuring prothrombin time (PT), thrombin time (TT), rotational thromboelastometry (ROTEM®), etc.

In some embodiments, the anticoagulant effects of an anti-FXI/FXIa antibody or antigen-binding fragment described herein (e.g., Antibody 1) are achieved by administering a therapeutically effective amount of recombinant activated factor VII (rFVIIa), e.g., a low dose. As used herein, a “low dose” of rFVIIa refers to a therapeutically effective amount of rFVIIa of less than about 15 μg/kg.

In certain embodiments, the therapeutically effective amount of rFVIIa is between about 0.5 μg/mL and about 14.5 μg/mL, e.g., between about 0.5 μg/mL and about 14 μg/mL, between about 0.5 μg/mL and about 13.5 μg/mL, between about 0.5 g/mL and about 13 μg/mL, between about 0.5 μg/mL and about 12.5 μg/mL, between about 0.5 μg/mL and about 12 μg/mL, between about 0.5 g/mL and about 11.5 μg/mL, between about 0.5 μg/mL and about 11 μg/mL, between about 0.5 μg/mL and about 10.5 μg/mL, between about 0.5 μg/mL and about 10 μg/mL, between about 0.5 μg/mL and about 9.5 μg/mL, between about 0.5 μg/mL and about 9 μg/mL, between about 0.5 μg/mL and about 8.5 μg/mL, between about 0.5 μg/mL and about 8 μg/mL, between about 0.5 μg/mL and about 7.5 μg/mL, between about 0.5 μg/mL and about 7 μg/mL, between about 0.5 μg/mL and about 6.5 μg/mL, between about 0.5 μg/mL and about 6 μg/mL, between about 0.5 μg/mL and about 5.5 μg/mL, between about 0.5 μg/mL and about 5 μg/mL, between about 0.5 g/mL and about 4.5 μg/mL, between about 0.5 μg/mL and about 4 μg/mL, between about 0.5 μg/mL and about 3.5 μg/mL, between about 0.5 μg/mL and about 3 μg/mL, between about 0.5 μg/mL and about 2.5 μg/mL, between about 0.5 μg/mL and about 2 μg/mL, between about 0.5 μg/mL and about 1.5 μg/mL, or 0.5 μg/mL and about 1 μg/mL. In certain embodiments, the therapeutically effective amount of rFVIIa is between about 0.5 μg/mL and about 1 μg/mL.

In certain embodiments, the therapeutically effective amount of rFVIIa is about 14.5 μg/mL, about 14 μg/mL, about 13.5 μg/mL, about 13 μg/mL, about 12.5 μg/mL, about 12 μg/mL, about 11.5 μg/mL, about 11 μg/mL, about 10.5 μg/mL, about 10 μg/mL, about 9.5 μg/mL, about 9 μg/mL, about 8.5 μg/mL, about 8 μg/mL, about 7.5 μg/mL, about 7 μg/mL, about 6.5 μg/mL, about 6 μg/mL, about 5.5 μg/mL, about 5 μg/mL, about 4.5 μg/mL, about 4 μg/mL, about 3.5 μg/mL, about 3 μg/mL, about 2.5 μg/mL, about 2 μg/mL, about 1.5 μg/mL, about 1 μg/mL, or about 0.5 μg/mL. In some embodiments, rFVIIa is administered at a dose of about 0.5 μg/mL. In some embodiments, rFVIIa is administered at a dose of about 1 μg/mL.

In some embodiments, the rFVIIa is administered once. In some embodiments, the rFVIIa is administered twice. In some embodiments, the rFVIIa is administered more than once, e.g., two, three, four, five or more times.

In some embodiments, the therapeutically effective amount of rFVIIa is administered as needed. “Administered as needed,” as used herein, refers to administering of rFVIIa until symptoms, e.g., bleeding, are treated and/or risk of bleeding has decreased. In some embodiments, the rFVIIa is administered for a certain duration of time, e.g., the time of a surgical procedure. In some embodiments, the rFVIIa is administered for a certain duration of time, e.g., the time of a surgical procedure and inpatient care. In some embodiments, the rFVIIa is administered as medically necessary, e.g., as determined by a physician.

In some embodiments, the rFVIIa is administered every hour. In some embodiments, the rFVIIa is administered every two hours. In some embodiments, the rFVIIa is administered every four hours. In some embodiments, the rFVIIa is administered every six hours. In some embodiments, the rFVIIa is administered every eight hours. In some embodiments, the rFVIIa is administered twice per day, e.g., every twelve hours. In certain embodiments, the frequency of rFVIIa administration decreases as the risk of bleeding decreases, e.g., the rFVIIa is administered every two hours for a set period of time (e.g., for 24 hours or 48 hours), then the rFVIIa is administered every four hours for a set period of time (e.g., up to a week).

In some embodiments, the therapeutically effective amount of recombinant activated factor VII (rFVIIa), e.g., a low dose, is administered intravenously to the subject, e.g., by intravenous bolus injection.

In some embodiments, the anticoagulant effects of an anti-FXI/FXIa antibody or antigen-binding fragment described herein (e.g., Antibody 1) are assessed using methods known in the art. For example and without limitation, anticoagulant effects may be determined by clotting time (CT), clot formation time (CFT), activated partial thromboplastin time (aPTT), etc. In certain embodiments, administering the therapeutically effective amount of rFVIIa prolongs CT in the subject treated with an anti-FXI/FXIa antibody or antigen-binding fragment described herein (e.g., Antibody 1) results in prolonged CT by at least 10%, at least 20%, at least 30%, at least 40%, at least 50%, at least 60%, at least 70%, at least 80%, or at least 90%, relative to CT prior to administering the rFVIIa. In certain embodiments, administering the therapeutically effective amount of rFVIIa prolongs CFT in the subject treated with an anti-FXI/FXIa antibody or antigen-binding fragment described herein (e.g., Antibody 1) results in prolonged CFT by at least 10%, at least 20%, at least 30%, at least 40%, at least 50%, at least 60%, at least 70%, at least 80%, or at least 90%, relative to CFT prior to administering the rFVIIa.

In some embodiments, the therapeutically effective amount of recombinant activated factor VII (rFVIIa) described herein can be administered in combination with one or more additional anticoagulant reversal therapies. Non-limiting examples of conventional strategies for reversing anticoagulant effects include (i) fluid replacement using colloids, crystalloids, human plasma or plasma proteins such as albumin, or (ii) transfusion with packed red blood or whole blood. Examples of therapies for reversal of the effects of anticoagulants, for example, in cases of severe emergency, include, without limitation, prohemostasis blood components such as fresh frozen plasma (FFP), prothrombin complex concentrates (PCC) and activated PCC (APCC), e.g. factor VIII inhibitor bypass activity (FEIBA). In some embodiments, the therapeutically effective amount of rFVIIa is administered in combination with an anticoagulant reversal therapy, such as emicizumab (ACE910), tranexamic acid, Fresh Frozen Plasma (FFP), Hemoeleven, Prothrombin Complex Concentrate (PCC), Activated PCC, or FEIBA (a FVIII inhibitor complex).

In some embodiments, the therapeutically effective amount of recombinant activated factor VII (rFVIIa) described herein can be administered in combination with an anti-idiotypic antibody, or antigen-binding fragment thereof, to an anti-FXI/FXIa antibody or antigen-binding fragment described herein (e.g., Antibody 1). Methods of making anti-idiotypic antibodies are known in the art.

### Antiplatelet Combination Therapies

In one aspect, provided herein is a combination method of treating a subject, said method comprising administering an isolated anti-Factor XI (FXI) and/or anti-activated Factor XI (FXIa) antibody or an antigen-binding fragment thereof described herein, e.g., Antibody 1, at a dose of about 150 mg, and administering a therapeutically effective dose of an oral antiplatelet therapy. In some embodiments, the subject is afflicted with or at risk of developing a thromboembolic disorder. Exemplary antiplatelet therapies include, for example and without limitation, aspirin, clopidogrel, prasugrel, dipyridamole, ticlopidine, eptifibatide, and ticagrelor. In some embodiments, the antiplatelet therapy is aspirin. In some embodiments, the antiplatelet therapy is clopidogrel. In some embodiments, the antiplatelet therapy is prasugrel. In some embodiments, the antiplatelet therapy is dipyridamole. In some embodiments, the antiplatelet therapy is ticlopidine. In some embodiments, the antiplatelet therapy is eptifibatide. In some embodiments, the antiplatelet therapy is ticagrelor.

In one aspect, provided herein is a combination method of treating a subject, e.g., a subject afflicted with or at risk of developing a thromboembolic disorder, said method comprising administering an isolated anti-Factor XI (FXI) and/or anti-activated Factor XI (FXIa) antibody, or an antigen-binding fragment thereof described herein, e.g., Antibody 1, at a dose of about 125 nM, about 250 nM, or about 500 nM, and administering about 1 mg/mL aspirin. In another aspect, provided herein is a combination method of treating a subject, e.g., a subject afflicted with or at risk of developing a thromboembolic disorder, said method comprising administering an isolated anti-Factor XI (FXI) and/or anti-activated Factor XI (FXIa) antibody, or an antigen-binding fragment thereof described herein, e.g., Antibody 1, at a dose of about 150 mg, and administering about 100 mg aspirin. In certain embodiments, the aspirin is administered orally, e.g., in a pill or tablet. In certain embodiments, the aspirin is a low-dose aspirin. In certain embodiments, the aspirin is a dose of about 75 mg, about 81 mg, about 100 mg, about 150 mg, about 162 mg, or about 200 mg. In certain embodiments, the aspirin is administered daily. In certain embodiments, the aspirin is administered twice a day. In certain embodiments, the anti-FXI/FXIa antibody or antigen-binding fragment described herein (e.g., Antibody 1) is administered intravenously. In certain embodiments, the anti-FXI/FXIa antibody or antigen-binding fragment described herein (e.g., Antibody 1) is administered subcutaneously. In certain embodiments, the anti-FXI/FXIa antibody or antigen-binding fragment described herein (e.g., Antibody 1) is administered intravenously in a first dose and administered subcutaneously in subsequent doses (e.g., second dose, third dose, fourth dose, fifth dose, sixth dose, etc.). In certain embodiments, the anti-FXI/FXIa antibody or antigen-binding fragment described herein (e.g., Antibody 1) is administered monthly.

In one aspect, provided herein is a combination method of treating a subject, e.g., a subject afflicted with or at risk of developing a thromboembolic disorder, said method comprising administering an isolated anti-Factor XI (FXI) and/or anti-activated Factor XI (FXIa) antibody, or an antigen-binding fragment thereof described herein, e.g., Antibody 1, at a dose of about 125 nM, about 250 nM, or about 500 nM, and administering about 3 μM ticagrelor. In another aspect, provided herein is a combination method of treating a subject, e.g., a subject afflicted with or at risk of developing a thromboembolic disorder, said method comprising administering an isolated anti-Factor XI (FXI) and/or anti-activated Factor XI (FXIa) antibody, or an antigen-binding fragment thereof described herein, e.g., Antibody 1, at a dose of about 150 mg and administering about 60 mg ticagrelor. In certain embodiments, the ticagrelor is administered orally, e.g., in a pill or tablet. In certain embodiments, the ticagrelor is a dose of about 60 mg, about 90 mg, or about 180 mg. In certain embodiments, ticagrelor is administered at a loading dose (e.g., about 180 mg), and subsequent administrations are at maintenance doses (e.g., about 60 mg or about 90 mg). In certain embodiments, the ticagrelor is administered daily. In certain embodiments, the ticagrelor is administered twice a day. In certain embodiments, the anti-FXI/FXIa antibody or antigen-binding fragment described herein (e.g., Antibody 1) is administered intravenously. In certain embodiments, the anti-FXI/FXIa antibody or antigen-binding fragment described herein (e.g., Antibody 1) is administered subcutaneously. In certain embodiments, the anti-FXI/FXIa antibody or antigen-binding fragment described herein (e.g., Antibody 1) is administered intravenously in a first dose and administered subcutaneously in subsequent doses (e.g., second dose, third dose, fourth dose, fifth dose, sixth dose, etc.). In certain embodiments, the anti-FXI/FXIa antibody or antigen-binding fragment described herein (e.g., Antibody 1) is administered monthly.

In some embodiments, the subject administered the anti-FXI/FXIa antibody or antigen-binding fragment described herein (e.g., Antibody 1), e.g., the subject afflicted with or at risk of developing a thromboembolic disorder, is receiving a dual antiplatelet therapy (DAPT). In certain embodiments, the DAPT comprises ticagrelor and aspirin. In some embodiments, the subject is administered about 3 μM ticagrelor, about 1 mg/mL aspirin, and an anti-FXI and/or anti-FXIa antibody as described herein, e.g., Antibody 1. In certain embodiments, the ticagrelor is administered orally, e.g., in a pill or tablet. In certain embodiments, the ticagrelor is administered daily. In certain embodiments, the aspirin is administered orally, e.g., in a pill or tablet. In certain embodiments, the aspirin is a low-dose aspirin. In certain embodiments, the aspirin is administered daily. In certain embodiments, the anti-FXI/FXIa antibody or antigen-binding fragment described herein (e.g., Antibody 1) is administered intravenously. In certain embodiments, the anti-FXI/FXIa antibody or antigen-binding fragment described herein (e.g., Antibody 1) is administered subcutaneously. In certain embodiments, the anti-FXI/FXIa antibody or antigen-binding fragment described herein (e.g., Antibody 1) is administered intravenously in a first dose and administered subcutaneously in subsequent doses (e.g., second dose, third dose, fourth dose, fifth dose, sixth dose, etc.). In certain embodiments, the anti-FXI/FXIa antibody or antigen-binding fragment described herein (e.g., Antibody 1) is administered monthly.

In some embodiments, administering of the anti-FXI/FXIa antibody or antigen-binding fragment described herein (e.g., Antibody 1) does not affect platelet aggregation in the subject as compared to the subject treated with only the antiplatelet therapy, e.g., only aspirin or only ticagrelor. Methods of assessing platelet aggregation are known in the art and include, for example and without limitation, impedance platelet aggregometry.

In some embodiments, e.g., when a subject, e.g., the subject administered the anti-FXI/FXIa antibody or antigen-binding fragment described herein (e.g., Antibody 1), is at high risk of bleeding (e.g., as discussed supra), the subject may stop receiving the antiplatelet therapies and be administered a therapeutically effective amount of recombinant activated factor VII (rFVIIa), as discussed supra.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1—Treatment of Patients with Vascular Graft Thrombosis

### Purpose and Rationale

This study was designed to test the effects of increasing concentrations of Antibody 1 on platelet aggregation following stimulation with collagen or thrombin receptor activating peptide-6 (TRAP-6) compared to vehicle and active control (abciximab, anti-GP2b3a) and to determine the effects of both agents on thrombin generation.

### Methods

Whole blood was obtained in EDTA and citrate tubes (6 healthy donors). Specimens were spiked with vehicle, Antibody 1 (250, 500, or 1000 nM), or abciximab (50 nM). Platelet aggregation was recorded following induction using collagen (1 μg/mL) or TRAP-6 (8 μM) using a multiplate impedance aggregometer. The area under the curve (AUC) for platelet aggregation was determined in arbitrary aggregation*time (AU*min). Thrombin generation was also measured at the above Antibody 1 and abciximab concentrations in platelet-rich plasma using Thrombinoscope CAT (Calibrated Automated Thrombogram; Stago CH S.A. and tissue factor reagent (1 μM); fluorescence was measured by an automated plate reader fluorometer.

### Results

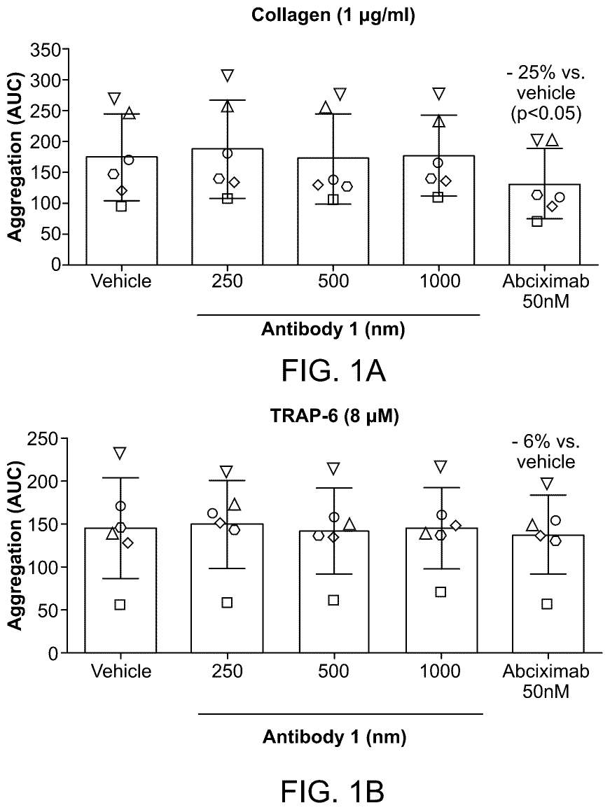

Antibody 1 showed no inhibitory or stimulatory effects on platelet aggregation induced by collagen (FIG. 1A) or TRAP-6 (FIG. 1B). In contrast, significant reductions in collagen-induced platelet aggregation were observed with abciximab. Antibody 1 resulted in significant delays in lag time and the time to peak concentration of thrombin generation; abciximab had no effect on thrombin generation.

Importantly, these results indicate that targeting FXI/FXIa by Antibody 1 does not affect platelet aggregation while inhibiting thrombin generation.

### Example 2—Antibody 1 does not Influence the Effects of Two Commonly Used Antiplatelet Agents In Vitro

### Purpose and Rationale

As demonstrated in Example 1 supra, Antibody 1 did not affect platelet function. Concomitant use of Antibody 1 with antiplatelet drugs was not previously evaluated. Therefore, this study was designed to assess the effects of increasing concentrations of Antibody 1 on the inhibition of platelet aggregation by aspirin or ticagrelor.

### Methods

Two studies were conducted concurrently. Blood specimens were obtained in EDTA and citrate tubes (6 healthy donors for each study). In the first study, whole blood was spiked with vehicle, aspirin (1 mg/mL), or aspirin (1 mg/mL)+Antibody 1 (125, 250, and 500 nM). Platelet aggregation was measured by impedance platelet aggregometry following induction with collagen (3.2 μg/mL) or arachidonic acid (0.5 mM) (Multiplate® analyzer, COLtest and ASPItest reagents; Roche Diagnostics International Ltd, Rotkreuz, Switzerland).

In the second study, whole blood was spiked with vehicle, ticagrelor (3 μM), or ticagrelor (3 μM)+Antibody 1 (125, 250, and 500 nM). Platelet aggregation was measured with the same methodology following induction by ADP (6.4 μM) or thrombin receptor activating peptide-6 (TRAP-6, 32 μM).

### Results

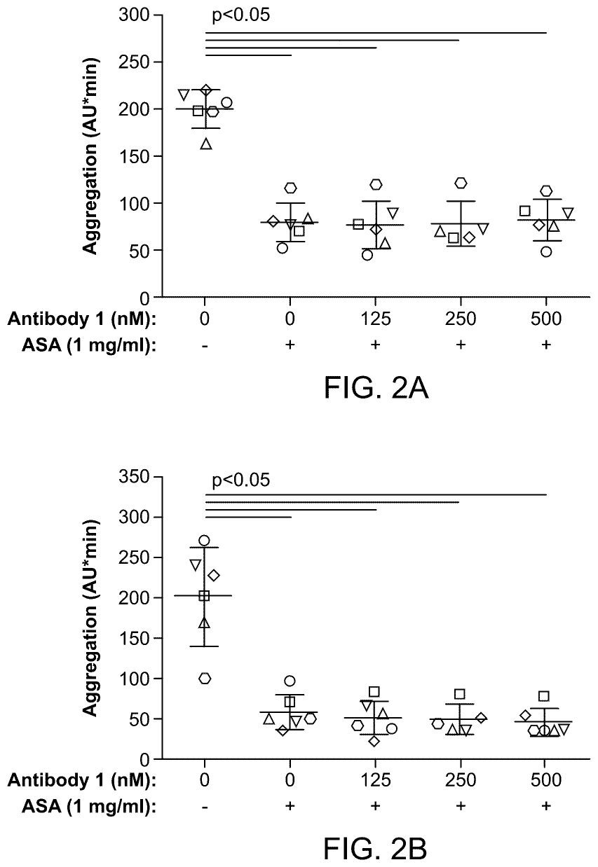

In the presence of aspirin, platelet aggregation induced with collagen (FIG. 2A) or arachidonic acid (FIG. 2B) was inhibited by 60% and 71%, respectively (p<0.05). Antibody 1 at 125, 250, and 500 ng/mL did not attenuate or increase the level of inhibition achieved with aspirin alone.

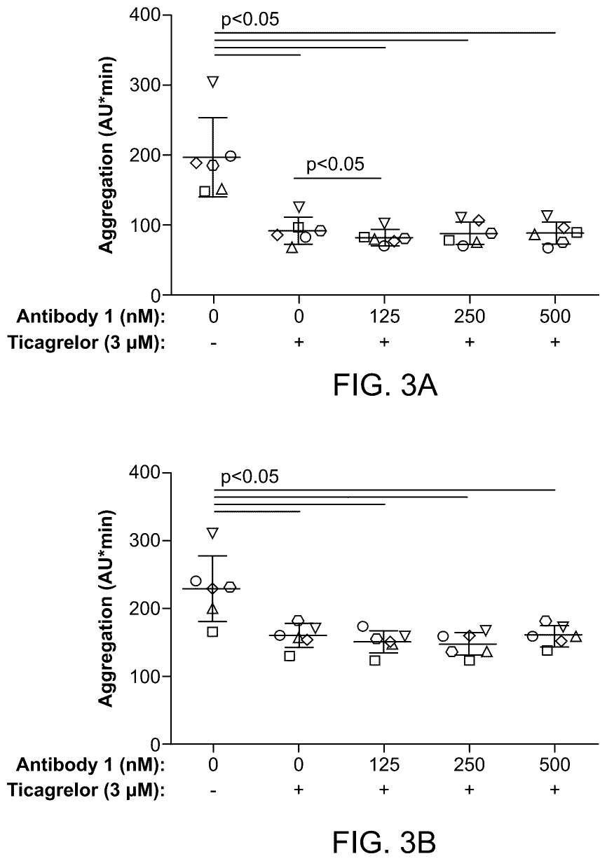

In the presence of ticagrelor, platelet aggregation induced with ADP (FIG. 3A) or TRAP-6 (FIG. 3B) was inhibited by 54% and 31%, respectively (p<0.05). Antibody 1 at 125, 250, and 500 ng/mL did not alter platelet inhibition achieved with ticagrelor alone.

### Conclusion

Antibody 1 at clinically relevant concentrations did not alter the inhibitory effects of aspirin or ticagrelor. These results suggest that pharmacodynamic interactions between Antibody 1 and aspiring or ticagrelor, two commonly used antiplatelet agents, are unlikely.

### Example 3—Low Concentrations of rFVIIa Bypass Changes in Rotational Thromboelastometry (ROTEM®) Coagulation Parameters Induced by Antibody 1 In Vitro

### Purpose and Rationale

Low doses of recombinant activated factor VII (rFVIIa) have been used to prevent or stop bleeding in patients with severe factor XI (FXI) deficiency. A similar strategy was hypothesized as a method to bypass the pharmacodynamic effects of Antibody 1. This study was designed to test whether low concentrations of rFVIIa could correct the changes in Antibody 1-induced coagulation parameters, as measured by rotational thromboelastometry (ROTEM) in a whole blood in vitro assay.

### Methods

Blood specimens were obtained in citrate tubes from 6 healthy human donors. Specimens were incubated with 15 and 30 μg/mL of Antibody 1 or vehicle for 10 minutes at 37° C. Specimens were subsequently spiked with rFVIIa at 0.5 and 1 μg/mL or vehicle control. Clot formation was monitored using ROTEM® delta analyzers and the non-activated thromboelastometry (NATEM) test using a recalcification reagent. CT (clotting time, in seconds), CFT (clot formation time, in seconds), alpha angle, etc. were measured and were compared to reference ranges provided by the manufacturer (Pentapharm GmbH, Munich, Germany).

### Results

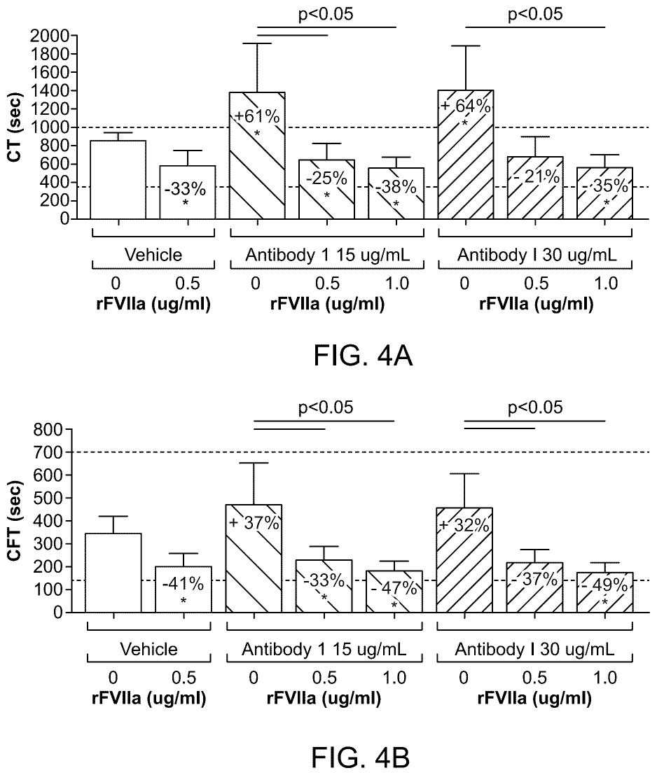

Antibody 1 at 15 and 30 μg/mL concentrations prolonged CT by 61% and 64%, respectively, prolonged CFT by 37% and 32%, respectively, and decreased alpha angle by 10% and 14% vs baseline, respectively. Adding rFVIIa at 0.5 and 1.0 μg/mL to Antibody 1-spiked samples shortened CT by 21% and 38% respectively, CFT by 33% and 49%, respectively, and increased alpha angle by 29% and 47%, respectively. CT parameters are presented in FIG. 4A, and CFT parameters are presented in FIG. 4B. NATEM parameters remained within normal reference ranges when rFVIIa was added.

### Conclusion

Low concentrations of rFVIIa (0.5 to 1 μg/mL) were able to correct the effects of Antibody 1 as assessed by rotational thromboelastometry. Further, normalizing without over-correcting ROTEM coagulation parameters suggested that tested concentrations of rFVIIa are unlikely to result in a hypercoagulable state. These data support using low doses of rFVIIa for bleeding management in patients treated with Antibody 1.

1. A method of reversing an anticoagulant effect of an isolated anti-Factor XI (FXI) and/or anti-activated Factor XI (FXIa) antibody or antigen-binding fragment thereof in a subject being administered the isolated antibody or antigen-binding fragment thereof at a dose of about 150 mg, wherein the method comprises administering to the subject a therapeutically effective amount of recombinant activated factor VII (rFVIIa), thereby reversing the anticoagulant effect.

2. (canceled)

3. The method of claim 1, wherein the therapeutically effective amount of rFVIIa is:
(a) between 0.5 and 1 μg/mL; and/or
(b) is administered once.

4-5. (canceled)

6. The method of claim 1, wherein the administering the therapeutically effective amount of rFVIIa prolongs clotting time (CT) and/or clot formation time (CFT) relative to CT or CFT, respectively, prior to the administering the therapeutically effective amount.

7. (canceled)

8. The method of claim 6, wherein the CT or CFT is determined by rotational thromboelastometry (ROTEM) in a whole blood assay.

9. (canceled)

10. The method of claim 1, wherein the method further comprises administering one or more of the following to the subject:
(a) fluid replacement using colloids, crystalloids, human plasma, or plasma proteins;
(b) transfusion with packed red blood or whole blood;
(c) fresh frozen plasma (FFP), prothrombin complex concentrates (PCC), or activated PCC (APCC); and
(d) an anti-idiotypic antibody against the isolated anti-FXI and/or anti-FXIa antibody or antigen-binding fragment thereof.

11. A method of treating a disease or disorder in a subject in need thereof, the method comprising administering to the subject a dose of about 150 mg of an isolated anti-Factor XI (FXI) and/or anti-activated Factor XI (FXIa) antibody or antigen-binding fragment thereof and
(a) about 1 mg/mL aspirin; or
(b) about 3 μM ticagrelor.

12. The method of claim 11, wherein the aspirin or ticagrelor is administered prior to, concurrently, or after the administering of the antibody or antigen-binding fragment thereof.

13-14. (canceled)

15. The method of claim 11, wherein the administering of the antibody or antigen-binding fragment thereof does not affect platelet aggregation in the subject as compared to the subject treated with only aspirin or ticagrelor.

16. The method of claim 15, wherein the platelet aggregation is measured by impedance platelet aggregometry.

17. The method of claim 16, wherein the platelet aggregation is induced by collagen, adenosine 5′-diphosphate (ADP), or thrombin receptor activating peptide-6 (TRAP-6).

18-19. (canceled)

20. The method of claim 1, wherein the antibody or antigen-binding fragment thereof comprises:
(a) a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39;
(b) a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
(c) a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 amino acid sequence of KNY; and a light chain variable region CDR3 of SEQ ID NO: 38;
(d) a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region amino acid sequence of KNY; and a light chain variable region CDR3 of SEQ ID NO: 15; and/or
(e) a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

21. The method of claim 1, wherein:
(a) the antibody or antigen-binding fragment thereof comprises a VH selected from the group consisting of SEQ ID NOs: 9, 29, and a VH with at least 90% identity thereto; and a VL selected from the group consisting of SEQ ID NOs: 19, 39, and a VL with at least 90% identity thereto; and/or
(b) the antibody comprises a heavy chain comprising an amino acid sequence selected from the group consisting of SEQ ID NOs: 31, 11, and a heavy chain with at least 90% identity thereto; and a light chain comprising an amino acid sequence selected from the group consisting of SEQ ID NOs: 41, 21, and a light chain with at least 90% identity thereto.

22-24. (canceled)

25. The method of claim 1, wherein the antibody:
(a) is a human monoclonal antibody;
(b) is a human IgG1 isotype; and/or
(c) the antibody comprises D265A and P329A substitutions in the Fc domain.

26-27. (canceled)

28. The method of claim 1, wherein the antibody or antigen-binding fragment thereof is administered intravenously or subcutaneously.

29. (canceled)

30. The method of claim 1, wherein the antibody or antigen-binding fragment thereof is administered in a drug delivery formulation comprising:
(a) a histidine buffer at a concentration of about 20 mM;
(b) sucrose at a concentration of about 220 mM;
(c) polysorbate-20 at a concentration of about 0.04% (v/v); and/or
(d) a diluent comprising about 5% (w/v) glucose,
wherein the drug delivery formulation is at pH 5.5.

31-34. (canceled)

35. The method of claim 1, wherein the subject is afflicted with or at risk of developing a thromboembolic disorder.

36. The method of claim 35, wherein the thromboembolic disorder is selected from the group consisting of: atrial fibrillation or atrial flutter, transient ischemic attack, ischemic stroke, thromboembolic stroke, hemorrhagic stroke, venous thromboembolism (VTE), pediatric VTE, systemic embolism, non-central nervous systemic embolism, myocardial infarction, deep vein thrombosis, severe protein S deficiency, cerebrovascular accident, and cancer.

37. The method of claim 1, wherein:
(a) the antibody or antigen-binding fragment thereof is administered about once a month and/or
(b) a first dose of the antibody or antigen-binding fragment thereof is administered intravenously and a second dose of the antibody or antigen-binding fragment is administered subcutaneously.

38. The method of claim 37, further comprising one or more additional doses of the antibody or antigen-binding fragment thereof administered subcutaneously following the administering of the second dose.

39. (canceled)


SEQUENCE LISTING (48 sequences)

SEQ ID NO: 1 - protein, 625 aa, Homo sapiens [sequence omitted]

SEQ ID NO: 2 - DNA, 3278 bp, Homo sapiens
  source 1..3278: mol_type = genomic DNA
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 3 - protein, 5 aa, synthetic construct
TAAMS

SEQ ID NO: 4 - protein, 17 aa, synthetic construct
GISGSGSSTYYADSVKG

SEQ ID NO: 5 - protein, 13 aa, synthetic construct
ELSYLYSGYYFDY

SEQ ID NO: 6 - protein, 7 aa, synthetic construct
GFTFSTA

SEQ ID NO: 7 - protein, 6 aa, synthetic construct
SGSGSS

SEQ ID NO: 8 - protein, 13 aa, synthetic construct
ELSYLYSGYYFDY

SEQ ID NO: 9 - protein, 122 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSS

SEQ ID NO: 10 - DNA, 366 bp, synthetic construct
  source 1..366: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 11 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1356 bp, synthetic construct
  source 1..1356: mol_type = other DNA
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 13 aa, synthetic construct
SGSSSNIGSNDVS

SEQ ID NO: 14 - protein, 7 aa, synthetic construct
KNYNRPS

SEQ ID NO: 15 - protein, 11 aa, synthetic construct
SAWDQRQFDVV

SEQ ID NO: 16 - protein, 9 aa, synthetic construct
SSSNIGSND

SEQ ID NO: 17 - skipped

SEQ ID NO: 18 - protein, 8 aa, synthetic construct
WDQRQFDV

SEQ ID NO: 19 - protein, 110 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 20 - DNA, 330 bp, synthetic construct
  source 1..330: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtccta

SEQ ID NO: 21 - protein, 216 aa, synthetic construct
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - DNA, 648 bp, synthetic construct
  source 1..648: mol_type = other DNA
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 23 - protein, 5 aa, synthetic construct
TAAMS

SEQ ID NO: 24 - protein, 17 aa, synthetic construct
GISGSGSSTYYADSVKG

SEQ ID NO: 25 - protein, 13 aa, synthetic construct
ELSYLYSGYYFDY

SEQ ID NO: 26 - protein, 7 aa, synthetic construct
GFTFSTA

SEQ ID NO: 27 - protein, 6 aa, synthetic construct
SGSGSS

SEQ ID NO: 28 - protein, 13 aa, synthetic construct
ELSYLYSGYYFDY

SEQ ID NO: 29 - protein, 122 aa, synthetic construct; identical to SEQ ID NO: 9

SEQ ID NO: 30 - DNA, 366 bp, synthetic construct
  source 1..366: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagc

SEQ ID NO: 31 - protein, 452 aa, synthetic construct
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1356 bp, synthetic construct
  source 1..1356: mol_type = other DNA
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 33 - protein, 13 aa, synthetic construct
SGSSSNIGSNDVS

SEQ ID NO: 34 - protein, 7 aa, synthetic construct
KNYNRPS

SEQ ID NO: 35 - protein, 11 aa, synthetic construct
SAWDQRQFDVV

SEQ ID NO: 36 - protein, 9 aa, synthetic construct
SSSNIGSND

SEQ ID NO: 37 - skipped

SEQ ID NO: 38 - protein, 8 aa, synthetic construct
WDQRQFDV

SEQ ID NO: 39 - protein, 110 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVL

SEQ ID NO: 40 - DNA, 330 bp, synthetic construct
  source 1..330: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctg

SEQ ID NO: 41 - protein, 216 aa, synthetic construct
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 648 bp, synthetic construct
  source 1..648: mol_type = other DNA
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 43 - protein, 8 aa, synthetic construct
GFTFSTAA

SEQ ID NO: 44 - protein, 8 aa, synthetic construct
ISGSGSST

SEQ ID NO: 45 - protein, 15 aa, synthetic construct
ARELSYLYSGYYFDY

SEQ ID NO: 46 - protein, 10 aa, synthetic construct
GFTFSTAAMS

SEQ ID NO: 47 - protein, 8 aa, synthetic construct
SSNIGSND

SEQ ID NO: 48 - protein, 6 aa, synthetic construct
HHHHHH